(19) United States
(12) Patent Application Publication
(10) Pub. No.: US 2009/0137653 A1
(43) Pub. Date: May 28, 2009

(54) SUBSTITUTED BENZYLAMINES AS CYP2A INHIBITORS AND USES THEREOF TO TREAT NICOTINE DEPENDENCE

(75) Inventors: Omar Ghosheh, Richmond (CA); Jeffery R. Raymond, Vancouver (CA)

Correspondence Address: SEED INTELLECTUAL PROPERTY LAW GROUP PLLC, 701 FIFTH AVE, SUITE 5400, SEATTLE, WA, 98104, US

(73) Assignee: INFLAYZME PHARMACEUTICALS LTD., Richmond, BC (CA)

(21) Appl. No.: 11/910,661

(22) PCT Filed: Apr. 6, 2006
(86) PCT No.: PCT/US2006/013081

§ 371 Date: Nov. 24, 2008

Related U.S. Application Data
(60) Provisional application No. 60/669,038, filed on Apr. 6, 2005.

(51) Int. Cl. A61K 31/417; C07C 211/27; C07D 233/46; A61P 25/32; A61K 31/137

(52) U.S. Cl. 514/398; 564/384; 514/655; 548/326.5

(57) ABSTRACT

This invention is directed to substituted benzylamines which are useful as inhibitors of the CYP2A6 enzyme. Pharmaceutical compositions comprising the compounds and methods of using the compounds to treat nicotine dependence are also disclosed.

### FIELD OF THE INVENTION

The invention relates to substituted benzylamines, compositions including these benzylamines and their use in treating nicotine dependence.

### BACKGROUND OF THE INVENTION

### Nicotine and Tobacco Dependence

An estimated 1.2 billion people smoke tobacco worldwide (˜49 million in US) causing more than 4 million related deaths annually (430,000 in US). In addition, a larger number of people suffer from smoking-related cancers, cardiovascular and respiratory diseases. Smoking is inflicting enormous losses, hundreds of billions of dollars in annual health related costs worldwide (>150 billion in US annually). Nicotine is the primary component of tobacco responsible for dependence on cigarette smoking. Pharmacological and behavioral processes that determine tobacco addiction are similar to those that determine addiction to drugs such as heroin and cocaine. Nicotine addiction is believed to result mainly from increased release of dopamine in the region of nucleus acumbens by acting on the nicotinic receptors within the ventral tegmental area. The role of mesolimbic dopamine system in nicotine reward has been established. Chronic nicotine administration is thought to lead to structural and functional changes of the brain. As well as its effects on behavior, nicotine can increase blood pressure and heart rate and can decrease skin temperature. Smoking is a complex regulated behavior that is influenced by both genetic and environmental factors. Nicotine-dependent individuals regulate their smoking to maintain nicotine levels in their brain and plasma. Abrupt smoking cessation could lead to withdrawal symptoms such as dysphoric or depressed mood, insomnia, irritability, frustration and anger, difficulty concentrating, restlessness, decreased heart rate and increased appetite and weight gain. Such symptoms arising from abrupt smoking cessation, play a major role in the failure of such quitting attempts.

Dependence on nicotine is related to a rapid rise of nicotine concentrations in the arterial plasma. Nicotine reaches the brain in an estimated 7-19 seconds after inhalation of the smoke from a cigarette. The rapid delivery of nicotine to the brain causes the nicotinic receptors in the brain to experience a unique pattern of exposure, which is associated with strong drug reinforcement. Smokers titrate their smoking to ensure that brain and plasma nicotine levels are maintained within the individual's target concentration. The half-life of nicotine is 1-2 hours.

### Nicotine Metabolism and CYP2A6

Nicotine is mainly metabolized in the liver, where around 70% of the drug is eliminated by first pass effect when taken orally. More than 20 metabolites of nicotine have been identified, all of which seem to be less pharmacologically active than the parent compound. The primary metabolite of nicotine in plasma, cotinine, has a half-life of 15 to 20 hours and concentrations exceed nicotine by 10-fold. Seventy to ninety percent of nicotine is metabolized through the enzyme known as CYP2A6 to cotinine (forming 5′-hydroxynicotine followed by oxidation by aldehyde oxidase, see FIG. 1 below). In addition, CYP2B6 plays a minor role in the formation of cotinine.

CYP2A6 is found mainly in liver with only traces in other parts of the body. Humans and monkeys have the CYP2A6 isoform but it has not yet been identified in other animals. Closely related isoforms such as CYP2A5 and CYP2A3 are found in other animals. In humans, CYP2A6 constitutes around 5% (range 1-10%) of the total abundance of CYP in liver and it is involved in metabolism of about 1-2% of drugs. Only a small number of clinically relevant drugs, namely nicotine and SM-12502, are mainly metabolized by CYP2A6. Other drugs metabolize through this enzyme only as a minor route of its elimination. CYP2A6 is also involved in activating many procarcinogens, especially the potent tobacco carcinogens such as 4-(methylnitrosamino)-1-(3-pyridyl)-1-butanone (NNK). The most potent recognized CYP2A6 inhibitors are methoxsalen, tranylcypromine and methofuran. As induction is concerned, it was reported that the enzyme could be induced in vitro by phenobarbital and rifampicin.

Genetic polymorphism in the CYP2A6 is well established. Seventeen genetic variants have been identified leading to different extents of metabolizing activity. Extensive metabolizers are those with two functional copies of the gene (wild type). Slow metabolizers are those with one or fewer active alleles. Fast metabolizers are carriers of three or more active gene copies. Poor metabolizers are carriers of defective or non-functional alleles. The frequency of the inactive alleles is ethnically diverse; it is low in European population, while a relatively high allele frequency of CYP2A6 gene deletion has been found in Asians. The link between CYP2A6 genetics and smoking behavior is established. Genetics explains ˜50% of variation in the initiation of smoking, ˜70% of the variation in maintenance and more than 80% of the variation in number of cigarettes smoked. Correlation with lower lung cancer susceptibility of individuals with defective CYP2A6 has been proposed.

### Treatment of Tobacco Dependence

Estimates suggest that in the US 70% of smokers would like to quit and 41% try to quit each year. The spectrum of available smoking cessation intervention can be classified into behavioral, pharmacological and alternative methods as follows: (1) Behavioral interventions, including counseling (group, individual, telephone), physician contact, non-physician and self-help showed to be reasonably productive (odds ratio 1.2-2.2, odds ratio is calcuated as the multiple of the “no-treatment (placebo) response”). (2) Drug Interventions (average odds ratio to quit is ˜1.7, close to many behavioral interventions). Two types of pharmacological therapies are approved as first line drugs for smoking cessation by the FDA: NRT (patch, gum, lozenge, inhaler and nasal spray) and Zyban® (Bupropion, also used as antidepressant). These drugs tend to have efficacy in initial stages of withdrawal, however first year relapse rates are as high as 80%. The second line drugs such as Clonidine (antihypertensive), Nortriptyline (antidepressant) and Mechamylamine are not FDA approved specifically for smoking cessation treatment but showed to have positive effect in some studies. Alternative interventions (hypnosis, aversive therapy, Lobeline, etc.) do not have enough evidence to support their use for treatment of tobacco dependence. Existing Nicotine Replacement Therapy (NRT) such as nicotine gum or patch could fail due to the following: (1) extent of nicotine replacement is inadequate compared to the nicotine obtained from cigarette smoking; (2) NRT does not mimic the rapid rise and fall in the plasma nicotine concentration that is achieved by smoking cigarettes; (3) wide variation of nicotine metabolism among individuals results in wide variation in plasma levels; (4) dermal or gastric irritation from use of NRT may limit their potential use; (5) difficult to overcome habits of smoking such as the feel of having a cigarette in hand or mouth; and (6) other factors make important contributions to the maintenance of smoking (conditional responses and environmental cues).

### CYP2A6 Inhibitors as Treatment of Tobacco Dependence

The director of the National Institute on Drug Abuse (NIDA) has acknowledged that CYP2A6 inhibitor research “opens up an exciting new avenue of treatment that can help smokers substantially reduce their exposure to the deadly particles of tobacco smoke while they overcome the addiction to nicotine that makes it so hard to quit”.

Inhibition of CYP2A6 and slower nicotine metabolism was proven clinically to have the following effects: (1) decrease risk of smoking initiation and dependence; (2) decrease in amount smoked; (3) decrease risk of tobacco related cancers and mutations and (4) combining nicotine with CYP2A6 inhibitor decreased smoking. Methoxsalen and tranylcypromine are drugs approved in market for other indications; methoxsalen as antineoplastic and antipsoriatic, and tranylcypromine as antidepressant. Both drugs are potent CYP2A6 inhibitors (CYP2A6 Ki=0.2 uM in human liver microsomes) with considerable selectivity (methoxsalen also interacts strongly with CYP1A2, Ki=1.0 uM), however, both have many side effects and short half lives (1-3 hours) that limit their use for treating tobacco dependence. Nevertheless, both drugs have proven the concept of the utilization of CYP2A6 inhibitors for this intended use.

### REFERENCES

The following is a list of references to support the above background information and use of the described invention:
Ariyoshi N, Miyamoto M, Umetsu Y, Kunitoh H, Dosaka-Akita H, Sawamura Y, Yokota J, Nemoto N, Sato K and Kamataki T. (2002) Genetic polymorphism of CYP2A6 gene and tobacco-induced lung cancer risk in male smokers. Cancer Epidemiol Biomarkers Prev. 11(9):890-894.
Cryan J F, Gasparini F, van Heeke G and Markou A. (2003) Non-nicotinic neuropharmacological strategies for nicotine dependence: beyond bupropion. Drug Discov Today. 8(22):1025-1034.
Glover E D, Glover P N and, Payne T J (2003) Treating nicotine dependence. Am J Med. Sci. 326(4):183-186.
Kamataki T, Nunoya K, Sakai Y, Kushida H and Fujita K (1999) Genetic polymorphism of CYP2A6 in relation to cancer. Mutat Res. 428(1-2):125-130.
Marlow S P, and Stoller J K (2003) Smoking Cessation. Respir Care 48(12):1238-1256.
MMWR-Annual Smoking (2002) Attributable Mortality, Years of Potential Life Lost and Economics Costs—United States 1995-1999 Apr. 12, 2002 Vol. 51, No. 14.
NIDA News Release Jul. 26, 2000. Medication Reduces Metabolism of Nicotine, Decreasing Urge to Smoke.
Oscarson M (2001) Genetic polymorphisms in the cytochrome P450 2A6 (CYP2A6) gene: implications for interindividual differences in nicotine metabolism. Drug Metab Dispos. 29(2):91-95.
Pianezza M L, Sellers E M and Tyndale R F (1998) Nicotine metabolism defect reduces smoking. Nature. 393(6687):750.
Sellers E M, Kaplan H L and Tyndale R F (2000) Inhibition of cytochrome P450 2A6 increases nicotine's oral bioavailability and decreases smoking. Clin Pharmacol Ther. 68(1):35-43.
Sellers E M, Ramamoorthy Y, Zeman M V, Djordjevic M V and Tyndale R F (2003 b) The effect of methoxsalen on nicotine and 4-(methylnitrosamino)-1-(3-pyridyl)-1-butanone (NNK) metabolism in vivo. Nicotine Tob Res. 5(6):891-899.
Sellers E M, Tyndale R F and Fernandes L C (2003 a) Decreasing smoking behaviour and risk through CYP2A6 inhibition Drug Discov Today 8 (11): 487-493.
Surgeon General Report (1998) The Health Consequences of Smoking.
Tomkins D M and Sellers E M (2001) Addiction and the brain: the role of neurotransmitters in the cause and treatment of drug dependence. CMAJ 164(6):817-821.
Tyndale R F and Sellers E M (2002) Genetic variation in CYP2A6-mediated nicotine metabolism alters smoking behavior. Ther Drug Monit. 24(1):163-71.
Xu C, Goodz S, Sellers E M and Tyndale R F (2002) CYP2A6 genetic variation and potential consequences. Adv Drug Deliv Rev. 54(10):1245-1256.
Zhang W, Kilicarslan T, Tyndale R F and Sellers E M (2001) Evaluation of methoxsalen, tranylcypromine, and tryptamine as specific and selective CYP2A6 inhibitors in vitro Drug Metab Dispos. 29(6):897-902.

These references and others cited herein, may be used by one skilled in the art to appreciate the scope and intent of this invention. The disclosures of these references are incorporated in full herein by reference in their entireties.

### SUMMARY OF THE INVENTION

In one aspect, the present invention provides methods of using compounds of formula (I) in treating nicotine dependence:

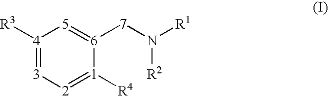

as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;

wherein:

R¹ and R² are independently selected from H, optionally substituted aryl, optionally substituted cycloalkyl, and optionally substituted C₁₋₃₀alkyl;

or R¹ and R², together with the nitrogen to which they are attached, form an optionally substituted heterocyclyl ring;

R³ is independently selected from X, optionally substituted C₁₋₃₀alkyl, C₁₋₃₀haloalkyl, nitro, carboxyl, —S(O)₂R, and —S(O)₂N(R)₂;

R⁴ is independently selected from H, X, optionally substituted C₁₋₃₀alkyl, C₁₋₃₀haloalkyl, —OR, —SR and —N(R)₂;

or R⁴ and R², together with the carbon and nitrogen to which they are attached, form an optionally substituted fused heterocyclyl ring;

where the numerals 1 through 7 in the formula (I) represent carbon atoms;

where the carbon atoms at numerals 2, 3 and 5 are independently optionally substituted with —X, —R, —N(R)₂ or —OR;

and where the carbon atom at numeral 7 is independently substituted with:
a) one of the following: ═O, ═C(R)₂, ═C═C(R)₂, —C(R)₂[C(R)₂]ₙ— and —O[C(R)₂]O— wherein n is 1 to 6; or
b) two of the following, which are independently selected: —X, —R and —OR;

each X is independently fluoro, bromo, chloro or iodo; and

each R is independently selected from H and a C₁₋₃₀ organic moiety.

In another aspect, this invention provides specific compounds of formula (I) as set forth herein.

In another aspect, the present invention provides pharmaceutical compositions comprising a pharmaceutically acceptable carrier and a compound of formula (I) as defined above.

In another aspect, this invention provides pharmaceutical compositions comprising a pharmaceutically acceptable carrier and specific compounds of formula (I) as set forth herein.

In another aspect, the present invention relates to the treatment of conditions requiring a reduction in the activity of a human cytochrome P450 enzyme CYP2A by treatment of a subject in need thereof with a compound of the invention or a pharmaceutical composition of the invention which acts as an inhibitor of the CYP2A enzyme and especially as a selective inhibitor of the CYP2A6 isozyme of the CYP2A enzyme.

In another aspect of the invention, the compound or compounds either on their own or as part of a pharmaceutical composition are used in conjunction with well-known and available behavioral, pharmacological and alternative methods of smoking cessation or reduction intervention to increase the effectiveness or the success rate of the intervention.

Another aspect of the invention is a method of using the compounds of the invention or the pharmaceutical compositions of the invention to reduce exposure by a subject to the harmful components of tobacco smoke by reducing the amount smoked as well the load of carcinogens in the body by inhibiting metabolism of procarcinogen to carcinogens and redirecting its metabolism to non-carcinogenic metabolites.

Another aspect of the invention is a method of using a compound of the invention or a pharmaceutical composition of the invention in combination with an oral NRT, wherein the compound inhibits the first pass metabolism thereby allowing the use of the oral NRT without having to use a higher dose.

Another aspect of the invention is a method for inhibiting the activity of an isoform of a CYP2A enzyme comprising administering to a subject a therapeutically effective amount of a compound of the invention or a pharmaceutical composition the invention.

### DETAILED DESCRIPTION OF THE INVENTION

The invention relates to compounds useful as pharmaceutical agents for the treatment of nicotine dependence resulting from tobacco use. The compounds of the invention act as inhibitors of CYP2A enzymes in general and as selective inhibitors of the CYP2A6 isozyme in particular. Inhibition of the CYP2A6 isozyme by a compound or compounds of the invention slows the metabolism of nicotine to cotinine and results in a decreased need for the use of tobacco products per unit time required to maintain a particular target level of circulating nicotine.

### A. DEFINITIONS

In accordance with the present invention and as used herein, the following terms are defined to have the following meanings, unless explicitly stated otherwise:

As used herein the singular forms “a”, “and”, and “the” include plural referents unless the context clearly dictates otherwise. For example, “a compound” refers to one or more of such compounds, while “the enzyme” includes a particular enzyme as well as other family members and equivalents thereof as known to those skilled in the art. As used in the specification and appended claims, unless specified to the contrary, the following terms have the meaning indicated.

“Alkyl” refers to a straight or branched hydrocarbon chain radical consisting solely of carbon and hydrogen atoms, containing no unsaturation, having from one to thirty carbon atoms (C₁₋₃₀), preferably having from one to ten carbon atoms (C₁₋₁₀), more preferably having from one to eight carbon atoms (C₁₋₈), and which is attached to the rest of the molecule by a single bond, e.g., methyl, ethyl, n-propyl, 1-methylethyl (iso-propyl), n-butyl, n-pentyl, 1,1-dimethylethyl (t-butyl), and the like. Unless stated otherwise specifically in the specification, the alkyl radical may be optionally substituted by one or more substituents selected from the group consisting of halo, cyano, nitro, amidino, guanidino, ureido, —OR, —S(O)ₜR (where t is 0 to 2), —P(O)₂OR, —N(R)₂, —C(O)R, —OC(O)R, —C(O)OR, —C(O)N(R)₂ and —N(R)C(O)R where each R is independently hydrogen, alkyl, alkenyl, haloalkyl, cycloalkyl, cycloalkylalkyl, aryl, aralkyl, aralkenyl, heterocyclyl or heterocyclylalkyl. Unless stated otherwise specifically in the specification, it is understood that for radicals, as defined below, that contain a substituted alkyl group that the substitution can occur on any carbon of the alkyl group.

“Alkoxy” refers to a radical of the formula —ORₐ where Rₐ is an alkyl radical as defined above, e.g., methoxy, ethoxy, n-propoxy, 1-methylethoxy (iso-propoxy), n-butoxy, n-pentoxy, 1,1-dimethylethoxy (t-butoxy), and the like. Unless stated otherwise specifically in the specification, it is understood that for radicals, as defined below, that contain a substituted alkoxy group that the substitution can occur on any carbon of the alkoxy group. The alkyl radical in the alkoxy radical may be optionally substituted as described above.

“Alkenyl” refers to a straight or branched hydrocarbon chain radical consisting solely of carbon and hydrogen atoms, containing at least one double bond, having from two to thirty carbon atoms (C₂₋₃₀), preferably having from two to ten carbon atoms (C₂₋₁₀), more preferably having from two to eight carbon atoms (C₂₋₈), and which is attached to the rest of the molecule by a single bond or a double bond, e.g., ethenyl, prop-1-enyl, but-1-enyl, pent-1-enyl, penta-1,4-dienyl, and the like. Unless stated otherwise specifically in the specification, the alkenyl radical may be optionally substituted by one or more substituents selected from the group consisting of halo, cyano, nitro, amidino, guanidino, ureido, —OR, —S(O)ₜR (where t is 0 to 2), —P(O)₂OR, —N(R)₂, —C(O)R, —OC(O)R, —C(O)OR, —C(O)N(R)₂ and —N(R)C(O)R where each R is independently hydrogen, alkyl, alkenyl, haloalkyl, cycloalkyl, cycloalkylalkyl, aryl, aralkyl, aralkenyl, heterocyclyl or heterocyclylalkyl. Unless stated otherwise specifically in the specification, it is understood that for radicals, as defined below, that contain a substituted alkenyl group that the substitution can occur on any carbon of the alkenyl group.

“Alkynyl” refers to a straight or branched hydrocarbon chain radical consisting solely of carbon and hydrogen atoms, containing at least one triple bond, having from two to thirty carbon atoms (C₂₋₃₀), preferably having from two to ten carbon atoms (C₂₋₁₀), more preferably having from two to eight carbon atoms (C₂₋₈), and which is attached to the rest of the molecule by a single bond or a triple bond, e.g., ethynyl, prop-1-ynyl, but-1-ynyl, pent-1-ynyl, penta-1,4-diynyl, and the like. Unless stated otherwise specifically in the specification, the alkynyl radical may be optionally substituted by one or more substituents selected from the group consisting of halo, cyano, nitro, amidino, guanidino, ureido, —OR, —S(O)ₜR (where t is 0 to 2), —P(O)₂OR, —N(R)₂, —C(O)R, —OC(O)R, —C(O)OR, —C(O)N(R)₂ and —N(R)C(O)R where each R is independently hydrogen, alkyl, alkenyl, haloalkyl, cycloalkyl, cycloalkylalkyl, aryl, aralkyl, aralkenyl, heterocyclyl or heterocyclylalkyl. Unless stated otherwise specifically in the specification, it is understood that for radicals, as defined below, that contain a substituted alkynyl group that the substitution can occur on any carbon of the alkynyl group.

“Alkylene” or “alkylene chain” refer to a straight or branched divalent hydrocarbon chain consisting solely of carbon and hydrogen, containing no unsaturation and having from one to eight carbon atoms, e.g., methylene, ethylene, propylene, n-butylene, and the like. The alkylene chain may be optionally substituted by one or more substituents selected from the group consisting of halo, cyano, nitro, amidino, guanidino, ureido, —OR, —S(O)ₜR (where t is 0 to 2), —P(O)₂OR, —N(R)₂, —C(O)R, —OC(O)R, —C(O)OR, —C(O)N(R)₂ and —N(R)C(O)R where each R is independently hydrogen, alkyl, alkenyl, haloalkyl, cycloalkyl, cycloalkylalkyl, aryl, aralkyl, aralkenyl, heterocyclyl or heterocyclylalkyl. The alkylene chain may be attached to the rest of the molecule through any two carbons within the chain.

“Alkenylene” or “alkenylene chain” refers to a straight or branched divalent hydrocarbon chain consisting solely of carbon and hydrogen, containing at least one double bond and having from two to eight carbon atoms, e.g., ethenylene, prop-1-enylene, but-1-enylene, pent-1-enylene, hexa-1,4-dienylene, and the like. The alkenylene chain may be optionally substituted by one or more substituents selected from the group consisting of halo, cyano, nitro, amidino, guanidino, ureido, —OR, —S(O)ₜR (where t is 0 to 2), —P(O)₂OR, —N(R)₂, —C(O)R, —OC(O)R, —C(O)OR, —C(O)N(R)₂ and —N(R)C(O)R where each R is independently hydrogen, alkyl, alkenyl, haloalkyl, cycloalkyl, cycloalkylalkyl, aryl, aralkyl, aralkenyl, heterocyclyl or heterocyclylalkyl. The alkenylene chain may be attached to the rest of the molecule through any two carbons within the chain.

“Alkynylene” or “alkynylene chain” refers to a straight or branched divalent hydrocarbon chain consisting solely of carbon and hydrogen, containing at least one triple bond and optionally one double bond and having from two to eight carbon atoms, e.g., ethenylene, prop-1-enylene, but-1-enylene, pent-1-enylene, hexa-1,4-dienylene, and the like. The alkynylene chain may be optionally substituted by one or more substituents selected from the group consisting of halo, cyano, nitro, amidino, guanidino, ureido, —OR, —S(O)ₜR (where t is 0 to 2), —P(O)₂OR, —N(R)₂, —C(O)R, —OC(O)R, —C(O)OR, —C(O)N(R)₂ and —N(R)C(O)R where each R is independently hydrogen, alkyl, alkenyl, haloalkyl, cycloalkyl, cycloalkylalkyl, aryl, aralkyl, aralkenyl, heterocyclyl or heterocyclylalkyl. The alkynylene chain may be attached to the rest of the molecule through any two carbons within the chain.

“Amidino” refers to the radical —C(═NH)NH₂. Unless stated otherwise specifically in the specification, the amidino radical may be optionally substituted by one or more substituents selected from the group consisting of alkyl, alkenyl, aryl, aralkyl, aralkenyl, haloalkyl, cycloalkyl or cycloalkylalkyl.

“Aryl” refers to a phenyl or naphthyl radical. Unless stated otherwise specifically in the specification, the term “aryl” or the prefix “ar-” (such as in “aralkyl”) is meant to include aryl radicals optionally substituted by one or more substituents selected from the group consisting of alkyl, alkenyl, halo, haloalkyl, haloalkenyl, aryl, aralkyl, aralkenyl, cyano, nitro, amidino, guanidino, ureido, cycloalkyl, cycloalkylalkyl, cycloalkylalkenyl, —OR, —S(O)ₜR (where t is 0 to 2), —P(O)₂OR, —N(R)₂, —C(O)R, —OC(O)R, —C(O)OR, —C(O)N(R)₂ and —N(R)C(O)R where each R is independently hydrogen, alkyl, alkenyl, haloalkyl, cycloalkyl, cycloalkylalkyl, aryl, aralkyl, aralkenyl, heterocyclyl or heterocyclylalkyl.

“Aryloxy” refers to a radical of the formula —OR_{b} where R_{b} is an optionally substituted aryl group as defined above.

“Aralkyl” refers to a radical of the formula —RₐR_{b} where Rₐ is an alkyl radical as defined above and R_{b} is one or more aryl radicals as defined above, e.g., benzyl, diphenylmethyl and the like. The aryl radical(s) may be optionally substituted as described above.

“Aralkoxy” refers to a radical of the formula —ORₐR_{b} where Rₐ is an alkyl radical as defined above and R_{b} is one or more optionally substituted aryl radicals as defined above.

“Aralkenyl” refers to a radical of the formula —R_{c}R_{b} where R_{c} is an alkenyl radical as defined above and R_{b} is one or more aryl radicals as defined above, e.g., 3-phenylprop-1-enyl, and the like. The aryl radical(s) and the alkenyl radical may be optionally substituted as described above.

“Cycloalkyl” refers to a stable monovalent monocyclic or bicyclic hydrocarbon radical consisting solely of carbon and hydrogen atoms, having from three to ten carbon atoms, and which is partially or fully saturated and attached to the rest of the molecule by a single bond, e.g., cyclopropyl, cyclobutyl, cyclopentyl, cyclohexyl, decalinyl and the like. Unless otherwise stated specifically in the specification, the term “cycloalkyl” is meant to include cycloalkyl radicals which are optionally substituted by one or more substituents independently selected from the group consisting of alkyl, alkenyl, aryl, aralkyl, aralkenyl, halo, haloalkyl, cyano, nitro, amidino, guanidino, ureido, cycloalkyl, —OR, —S(O)ₜR (where t is 0 to 2), —P(O)₂OR, —N(R)₂, —C(O)R, —OC(O)R, —C(O)OR, —C(O)N(R)₂ and —N(R)C(O)R where each R is independently hydrogen, alkyl, alkenyl, haloalkyl, cycloalkyl, cycloalkylalkyl, aryl, aralkyl, aralkenyl, heterocyclyl or heterocyclylalkyl.

“Cycloalkylalkyl” refers to a radical of the formula —RₐR_{d} where Rₐ is an alkyl radical as defined above and R_{d} is a cycloalkyl radical as defined above. The alkyl radical and the cycloalkyl radical may be optionally substituted as defined above.

“Cycloalkylalkenyl” refers to a radical of the formula —R_{c}R_{d} where R_{c} is an alkenyl radical as defined above and R_{d} is a cycloalkyl radical as defined above. The alkenyl radical and the cycloalkyl radical may be optionally substituted as defined above.

“Formyl” refers to the radical —C(O)H.

“Formylmethyl” refers to the radical —CH₂C(O)H.

“Guanidino” refers to the radical —N(H)C(═NH)NH₂. Unless stated otherwise specifically in the specification, the guanidino radical may be optionally substituted by one or more substituents selected from the group consisting of alkyl, alkenyl, aryl, aralkyl, aralkenyl, haloalkyl, cycloalkyl or cycloalkylalkyl.

“Halo” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, and the like, having from one to thirty carbon atoms (C₁₋₃₀), preferably having from one to ten carbon atoms (C₁₋₁₀), more preferably having from one to eight carbon atoms (C₁₋₈)

“Haloalkenyl” refers to an alkenyl radical, as defined above, that is substituted by one or more halo radicals, as defined above, e.g., 2,2-difluoroethenyl, 1,2-difluoroethenyl, 3-bromo-2-fluoropropenyl, and the like.

“Haloalkoxy” refers to a radical of the formula —OR_{c} where R_{c} is an haloalkyl radical as defined above, e.g., trifluoromethoxy, difluoromethoxy, trichloromethoxy, 2,2,2-trifluoroethoxy, 1,2-difluoroethoxy, 3-bromo-2-fluoropropoxy, 1, 2-dibromoethoxy, and the like.

“Heterocyclyl” or “heterocyclyl ring” refers to a stable 3- to 15-membered ring radical which consists of carbon atoms and from one to five heteroatoms selected from the group consisting of nitrogen, oxygen and sulfur. For purposes of this invention, the heterocyclyl radical may be a monocyclic, bicyclic or tricyclic ring system, which may include fused or bridged ring systems; and the nitrogen, carbon or sulfur atoms in the heterocyclyl radical may be optionally oxidized (i.e., substituted with oxygen); the nitrogen atom may be optionally quaternized; and the heterocyclyl radical may be aromatic or partially or fully saturated. Examples of such heterocyclyl radicals include, but are not limited to, azepinyl, acridinyl, benzimidazolyl, benzthiazolyl, benzothiadiazolyl, benzoxazolyl, benzodioxolyl, benzodioxinyl, benzopyranyl, benzopyranonyl, benzofuranyl, benzofuranonyl, benzothienyl (benzothiophenyl), benzotriazolyl, carbazolyl, cinnolinyl, decahydroisoquinolyl, dioxolanyl, furanyl, furanonyl, isothiazolyl, imidazolyl, imidazolinyl, imidazolidinyl, isothiazolidinyl, indolyl, indazolyl, isoindolyl, indolinyl, isoindolinyl, indolizinyl, isoxazolyl, isoxazolidinyl, morpholinyl, naphthyridinyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, oxiranyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydrofuryl, triazinyl, tetrahydropyranyl, thienyl, thiamorpholinyl, thiamorpholinyl sulfoxide, and thiamorpholinyl sulfone. Unless stated otherwise specifically in the specification, the term “heterocyclyl” is meant to include heterocyclyl radicals as defined above which are optionally substituted by one or more substituents selected from the group consisting of oxo, alkyl, alkenyl, halo, haloalkyl, haloalkenyl, nitro, cyano, amidino, guanidino, ureido, haloalkyl, haloalkoxy, aryl, aralkyl, aralkenyl, cycloalkyl, cycloalkylalkyl, heterocyclyl, heterocyclylalkyl, —OR, —S(O)ₜR (where t is 0 to 2), —P(O)₂OR, —OR, —C(O)R, —OC(O)R, —C(O)OR, —C(O)N(R)₂, —N(R)₂, and —N(R)C(O)R wherein each R is independently hydrogen, alkyl, alkenyl, alkynyl, aryl, aralkyl, aralkenyl, cycloalkyl, cycloalkylalkyl, cycloalkylalkenyl, haloalkyl, haloalkenyl, heterocyclyl, and heterocyclylalkyl. For purposes of this invention, a “fused heterocyclyl ring” “Heterocyclylalkyl” refers to a radical of the formula —RₐRₑ where Rₐ is an alkyl radical as defined above and Rₑ is a heterocyclyl radical as defined above, and if the heterocyclyl is a nitrogen-containing heterocyclyl, the heterocyclyl may be attached to the alkyl radical at the nitrogen atom. The heterocyclyl radical may be optionally substituted as defined above.

“Ureido” refers to the radical —N(H)C(═O)NH₂. Unless stated otherwise specifically in the specification, the ureido radical may be optionally substituted by one or more substituents selected from the group consisting of alkyl, alkenyl, aryl, aralkyl, aralkenyl, haloalkyl, cycloalkyl or cycloalkylalkyl.

As used herein, compounds which are “commercially available” may be obtained from standard commercial sources including Acros Organics (Pittsburgh Pa.), Aldrich Chemical (Milwaukee Wis.; www.aldrich.sial.com, including Sigma Chemical and Fluka), American Tissue Culture Collection (ATCC, Rockville, Md.), Apin Chemicals Ltd. (Milton Park UK), Avocado Research (Lancashire U.K.), BDH Inc. (Toronto, Canada), Bionet (Cornwall, U.K.), Chemservice Inc. (West Chester Pa.), Crescent Chemical Co. (Hauppauge N.Y.), Eastman Organic Chemicals, Eastman Kodak Company (Rochester N.Y.), EM Industries, Inc. (Hawthorne, N.Y.; www.emscience.com), Fisher Scientific Co. (Pittsburgh Pa.), Fisher Scientific Co. (Hampton, N.H.; www.fischer1.com), Fisons Chemicals (Leicestershire UK), Frontier Scientific (Logan Utah), ICN Biomedicals, Inc. (Costa Mesa Calif.), Key Organics (Cornwall U.K.), Lancaster Synthesis (Windham N.H.; www.lancaster.co.uk), Maybridge Chemical Co. Ltd. (Cornwall U.K.), Parish Chemical Co. (Orem Utah), Pfaltz and Bauer, Inc. (Waterbury Conn.), Polyorganix (Houston Tex.), Pierce Chemical Co. (Rockford Ill.), Praxair (Vancouver, B.C.), Riedel de Haen AG (Hannover, Germany), Spectrum Quality Product, Inc. (New Brunswick, N.J.), Steraloids Inc. (Newport, R.I.), TCI America (Portland Oreg.), Trans World Chemicals, Inc. (Rockville Md.), and Wako Chemicals USA, Inc. (Richmond Va.).

As used herein, “suitable conditions” for carrying out a synthetic step are explicitly provided herein or may be discerned by reference to publications directed to methods used in synthetic organic chemistry. The reference books and treatise set forth above that detail the synthesis of reactants useful in the preparation of compounds of the present invention, will also provide suitable conditions for carrying out a synthetic step according to the present invention.

As used herein, “methods known to one of ordinary skill in the art” may be identified through various reference books and databases. Suitable reference books and treatise that detail the synthesis of reactants useful in the preparation of compounds of the present invention, or provide references to articles that describe the preparation, include for example, “Synthetic Organic Chemistry”, John Wiley and Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley and Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases (the American Chemical Society, Washington, D.C., www.acs.org may be contacted for more details). Chemicals that are known but not commercially available in catalogs may be prepared by custom chemical synthesis houses, where many of the standard chemical supply houses (e.g., those listed above) provide custom synthesis services.

As used herein, the term C₁₋₃₀ organic moiety refers to a stable arrangement of atoms composed of at least one and not more than about the maximum carbon number set forth in the range, typically not more than about 30 carbon atoms, and any number of non-carbon atoms.

The C₁₋₃₀ organic moiety may be a saturated or unsaturated hydrocarbyl radical. A saturated hydrocarbyl radical is defined according to the present invention as any radical composed exclusively of carbon and hydrogen, where single bonds are exclusively used to join carbon atoms together. Thus, any stable arrangement of carbon and hydrogen atoms, having at least one carbon atom, is included within the scope of a saturated hydrocarbon radical according to the invention. Some specific terminology that may be used to refer to specific carbon atom arrangements will be discussed below.

The carbon atoms may form an alkyl group as defined herein. The carbon atoms may form a cycloalkyl group as defined herein. Additional groups within the scope of “cycloalkyl” as defined herein are polycycloalkyl groups, defined below.

A polycycloalkyl group is an arrangement of carbon atoms wherein at least one carbon atom is a part of at least two separately identifiable rings. The polycycloalkyl group may contain bridging between two carbon atoms, where bicyclo[1.1.0]butyl, bicyclo[3.2.1]octyl, bicyclo[5.2.0]nonyl, tricycl[2.2.1.0¹]heptyl, norbornyl and pinanyl are representative examples. The polycycloalkyl group may contain one or more fused ring systems, where decalinyl (radical from decalin) and perhydroanthracenyl are representative examples. The polycycloalkyl group may contain a spiro union, in which a single atom is the only common member of two rings. Spiro[3.4]octyl, spiro[3.3]heptyl and spiro[4.5]decyl are representative examples.

In addition, the saturated hydrocarbyl radical can be composed of any combination of two or more of the above, i.e., any combination of alkyl and cycloalkyl groups. Thus, the C₁₋₃₀ organic moiety may be an alkyl group (e.g., methyl) with a cycloalkyl (e.g., cyclohexyl) substituent, so that C₁₋₃₀ organic moiety is a cyclohexylmethyl group. As another example, the C₁₋₃₀ organic moiety may be a cyloalkyl group (e.g., cyclooctyl) having two alkyl substituents (e.g., a methyl and ethyl substituent), so that the C₁₋₃₀ organic moiety is a methylethylcyclooctyl group. As a final example, the C₁₋₃₀ organic moiety may be a cycloalkyl group with an alkyl substituent, where the alkyl substituent is substituted with a polycycloalkyl substituent.

As indicated above, the C₁₋₃₀ organic moiety may be an unsaturated hydrocarbyl radical. Such an C₁₋₃₀ organic moiety is defined as having a carbon arrangement as set forth above for saturated hydrocarbyl radicals, with the additional feature that at least one bond between any two carbon atoms is other than a single bond. An alkyl group containing at least one single double bond is referred to herein as an alkenyl group. An alkyl group containing at least one triple bond is referred herein to as an alkynyl group.

Likewise, the cycloalkyl group may have one or more double or triple bonds, and be included within the scope of an unsaturated hydrocarbyl radical according to the invention. Cycloalkenyl and cycloalkynyl are general names given to groups having a single carbon-based ring with a single double and triple bond in the ring, respectively. Cycloalkadienyl groups are cycloalkyl groups with two double bonds contained in the ring structure. The double bond may be exocyclic to the ring, e.g., a carbon atom of the ring may have a ═CH₂ group (i.e., a methylidene group) or higher homologue bonded to it.

A ring may be unsaturated to the extent of being aromatic, and still be included within the scope of an unsaturated hydrocarbyl radical. Thus, an aryl group as defined herein is included within the scope of such hydrocarbyl groups. As any combination of the above is also included within the scope of an unsaturated hydrocarbyl radical, aralkyl (C₁₋₃₀ organic moiety is an alkyl group with at least one aryl substituent, e.g., benzyl) and alkylaryl (C₁₋₃₀ organic moiety is an aryl ring with at least one alkyl substituent, e.g., tolyl) groups are included within the scope of C₁₋₃₀ organic moiety. C₆ aryls are a preferred component of organic moieties of the invention.

Also included within the scope of an C₁₋₃₀ organic moiety are those organic moieties that contain one or more heteroatoms. Heteroatoms according to the invention are any atom other than carbon and hydrogen. A preferred class of heteroatoms are naturally occurring atoms (other than carbon and hydrogen). Another preferred class are non-metallic (other than carbon and hydrogen). Another preferred class consists of boron, nitrogen, oxygen, phosphorous, sulfur, selenium and halogen (i.e., fluorine, chlorine, bromine and iodine, with fluorine and chlorine being preferred). Another preferred class consists of nitrogen, oxygen, sulfur and halogen. Another preferred class consists of nitrogen, oxygen and sulfur. Oxygen is a preferred heteroatom. Nitrogen is a preferred heteroatom.

For example, the C₁₋₃₀ organic moiety may be a hydrocarbyl radical as defined above, with at least one substituent containing at least one heteroatom. In other words, the C₁₋₃₀ organic moiety may be a hydrocarbyl radical as defined above, wherein at least one hydrogen atom is replaced with a heteroatom. For example, if the heteroatom is oxygen, the substituent may be a carbonyl group, i.e., two hydrogens on a single carbon atom are replaced by an oxygen, to form either a ketone or aldehyde group. Alternatively, one hydrogen may be replaced by an oxygen atom, in the form of an hydroxy, alkoxy, aryloxy, aralkyloxy, alkylaryloxy (where alkoxy, aryloxy, aralkyloxy, alkylaryloxy may be collectively referred to as hydrocarbyloxy), heteroaryloxy, —OC(O)R, ketal, acetal, hemiketal, hemiacetal, epoxy and —OSO₃M. The heteroatom may be a halogen. The heteroatom may be a nitrogen, where the nitrogen forms part of an amino (—NH₂, —NHR, —N(R)₂), alkylamido, arylamido, arylalkylamido, alkylarylamido, nitro, —N(R)SO₃M or aminocarbonylamide group. The heteroatom may be a sulfur, where the sulfur forms part of a thiol, thiocarbonyl, —SO₃M, sulfonyl, sulfonamide or sulfonhydrazide group. The heteroatom may be part of a carbon-containing substituent such as formyl, cyano, —C(O)OH, —C(O)OR, —C(O)OM, —C(O)R, —C(O)N(R)₂, carbamate, carbohydrazide and carbohydroxamic acid.

In the above exemplary heteroatom-containing substituents, R represents the remainder of the C₁₋₃₀ organic moiety and M represents proton or a metal ion. Preferred metal ions, in combination with a counterion, form physiologically tolerated salts. A preferred metal from which a metal ion may be formed include an alkali metal [for example, lithium (Li), sodium (Na), potassium (K), rubidium (Rb) and cesium (Cs)] an alkaline earth metal (for example, magnesium (Mg), calcium (Ca) and strontium (Sr)], or manganese (Mn), iron (Fe), zinc (Zn) or silver (Ag). An alkali metal or an alkaline earth metal are preferred M groups. Sodium, potassium, magnesium and calcium are preferred M groups. Sodium and potassium are preferred M groups.

Another class of C₁₋₃₀ organic moieties according to the invention are hydrocarbyl radicals as defined above, wherein at least one heteroatom is substituted for a carbon atom in the hydrocarbyl. One example of such organic moieties is the heterocyclyls defined herein. Another example of such organic moieties have a heteroatom bridging (a) the radical to which the organic moiety is bonded and (b) the remainder of the organic moiety. Examples include alkoxy, aryloxy, aralkoxy and alkylaryloxy radicals, which may collectively be referred to herein as hydrocarbyloxy radicals or moieties. Thus, —OR is an exemplary C₁₋₃₀ organic moiety of the invention (where R is the remainder of the C₁₋₃₀ organic moiety). Another example is —NHR (where R is the remainder of the C₁₋₃₀ organic moiety). Other examples include —R¹⁰—OR⁶ and —R¹⁰—N(R⁷)₂ where R⁶ and R⁷ are as defined above in the Summary of the Invention and R¹⁰ is a bond or a straight or branched alkylene, alkenylene or alkynylene chain.

While the C₁₋₃₀ organic moiety may have up to about 30 carbon atoms, preferred organic moieties of the invention have fewer than 30 carbon atoms, for example, up to about 25 carbon atoms, more preferably up to about 20 carbon atoms. The organic moiety may have up to about 15 carbon atoms, or up to about 12 or 10 carbon atoms. A preferred category of organic moieties has up to about 8 or 6 carbon atoms.

A preferred group of C₁₋₃₀ organic moieties of the invention is that group consisting of alkyl, alkenyl, alkynyl, aryl, aralkyl, aralkenyl, cycloalkyl, cycloalkylalkyl, cycloalkylalkenyl, haloalkyl, haloalkenyl, heterocyclyl, and heterocyclylalkyl.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein.

The term “prodrug” is also meant to include any covalently bonded carriers which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol and amine functional groups in the compounds of the invention and the like.

“Stable compound” and “stable structure” are meant to indicate a compound that is sufficiently robust to survive isolation to a useful degree of purity from a reaction mixture, and formulation into an efficacious therapeutic agent.

“Subject” as used herein includes humans and domestic animals, such as cats, dogs, swine, cattle, sheep, goats, horses, rabbits, and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Oxygen protecting group” refers to a radical which protects and maintains a hydroxy group during subsequent chemical reactions. Such groups include, but are not limited to, trialkylsilyl or diarylalkylsilyl (e.g., t-butyldimethylsilyl, t-butyldiphenylsilyl or trimethylsilyl), tetrahydropyranyl, benzyl, and the like. Protecting groups may be added or removed in accordance with standard techniques, which are well-known to those skilled in the art and as described herein. The use of protecting groups, particularly oxygen protecting groups, is described in detail in Green, T. W. and P. G. M. Wutz, Protective Groups in Organic Synthesis (1991), 2nd Ed., Wiley-Interscience.

“Leaving group initiator” refers to a radical which, together with the oxygen to which is it attached, forms a leaving group which is easily removed from the rest of the molecule upon attack by the appropriate nucleophile. The hydroxy radical is not a good leaving group and must therefore be converted to a group that does leave. One way is to protonate the hydroxy radical (to form a more acidic leaving group). Another is to convert the hydroxy to a reactive ester, most commonly, to a sulfonic ester. The sulfonic ester groups tosylate, brosylate, nosylate and mesylate are frequently used. Other leaving groups include oxonium ions, alkyl perchorates, ammonioalkanesulfonate esters, alkyl fluorosulfonates and the fluorinated compounds triflates and nonaflates.

As used herein, an “effective amount” or a “therapeutically effective amount” of a substance is that amount sufficient to affect a desired biological effect, such as beneficial results, including clinical results.

“Pharmaceutically acceptable carriers” for therapeutic use are well known in the pharmaceutical art, and are described, for example, in Remington's Pharmaceutical Sciences, Mack Publishing Co. (current edition). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. In addition, antioxidants and suspending agents may be used.

“Pharmaceutically acceptable salt” refers to salts of a compound of the invention derived from the combination of such compounds and a pharmaceutically acceptable organic or inorganic acid (acid addition salts) or a pharmaceutically acceptable organic or inorganic base (base addition salts) which retain the biological effectiveness and properties of the compounds of the present invention and which are not biologically or otherwise undesirable. The compounds of the invention described herein may be used in either the free base or salt forms, with both forms being considered as being within the scope intended herein. Pharmaceutically-acceptable salts of the compounds of the invention include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chloro-benzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc, aluminum, and other metal salts, such as but not limited to sodium hydrogen phosphate and disodium phosphate; and also including, but not limited to, salts of mineral acids, such as but not limited to hydrochloride and sulfates; and salts of organic acids, such as but not limited to acetates, lactates, malates, tartrates, citrates, ascorbates, succinates, butyrates, valerates and fumarates. Other examples of pharmaceutically acceptable salt include but not limited to those described in for example: “Handbook of Pharmaceutical Salts, Properties, Selection, and Use”, P. Heinrich Stahl and Camille G. Wermuth (Eds.), Published by VHCA (Switzerland) and Wiley-VCH (FRG), 2002.

“Therapeutically effective amount” refers to that amount of a compound of the invention which, when administered to a mammal, preferably a human, is sufficient to effect the desired physiological treatment of the condition treated. The amount of a compound of the invention which constitutes a “therapeutically effective amount” will vary depending on the compound, the condition and its severity, and the age of the mammal to be treated, but can be determined routinely by one of ordinary skill in the art having regard to his own knowledge and to this disclosure.

“Treating” or “treatment” as used herein covers the treatment of the condition of interest in a mammal, preferably a human, having the condition of interest, and includes:

(i) preventing the condition from occurring in a mammal, in particular, when such mammal is predisposed to the condition but has not yet been diagnosed as having it;

(ii) inhibiting the condition, i.e., arresting its development; or

(iii) relieving the condition, i.e., causing regression of the condition.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

The nomenclature used herein for the compounds of the invention is a modified form of the I.U.P.A.C. nomenclature system wherein the compounds are named herein as derivatives of the benzylamine moiety.

The compounds of the invention may, and typically do, exist as solids, including crystalline solids which can be crystallized from common solvents such as ethanol, N,N-dimethylformamide, water, or the like. The crystallization process may, depending on the crystallization conditions, provide various polymorphic structures. Typically, a more thermodynamically stable polymorph is advantageous to the commercial scale manufacture of a steroid compound of the invention, and is a preferred form of the compound.

Often, crystallizations produce a solvate of the compound of the invention. As used herein, the term “solvate” refers to an aggregate that comprises one or more compounds of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

As used herein, a “pharmaceutically acceptable solvate” refers to a solvate that retains the biological effectiveness and properties of the biologically active compounds of the invention. Examples of pharmaceutically acceptable solvates include, but are not limited to, water, isopropanol, ethanol, methanol, DMSO, EtOAc, acetic acid, and ethanolamine. It should be appreciated by those skilled in the art that solvated forms are equivalent to unsolvated forms and are intended to be encompassed within the scope of the present invention. Sykes, P. A., Guidebook to Mechanism in Organic Chemistry, 6th Ed (1986, John Wiley and Sons, N.Y.) is an exemplary reference that describe solvates.

### B. SPECIFIC COMPOUNDS OF THE INVENTION

Certain compounds of formula (I), as set forth above in the Summary of the Invention, are of particular interest to the invention.

One group of compounds of formula (I) of particular interest are those compounds of formula (I) wherein:

R¹ and R² are independently selected from H; optionally substituted phenyl; optionally substituted benzyl; optionally substituted cyclopentyl; optionally substituted cyclohexyl; optionally substituted decahydronaphthalenyl; optionally substituted cyclopropylmethyl; optionally substituted cyclohexylmethyl; C₁₋₈alkyl optionally substituted with halo, —OR or —N(R)₂; optionally substituted furanylmethyl; optionally substituted tetrahydrofuranylmethyl; optionally substituted indolylethyl; and optionally substituted imidazolylmethyl;

or R¹ and R², together with the nitrogen to which they are attached, form an optionally substituted pyrrolidinyl, piperidinyl or morpholinyl ring;

R³ is independently selected from X, optionally substituted C₁₋₈alkyl, C₁₋₈haloalkyl, nitro, carboxyl, —S(O)₂R, and —S(O)₂N(R)₂;

R⁴ is independently selected from H, X, optionally substituted C₁₋₈alkyl, C₁₋₈haloalkyl, —OR, —SR and —N(R)₂;

the numerals 1 through 7 in formula (Ia) represent carbon atoms;

the carbon atoms at numerals 2, 3 and 5 are independently optionally substituted with —X, —R or —OR;

the carbon atom at numeral 7 is optionally substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;

each X is independently selected from fluoro, bromo, chloro or iodo; and

each R is independently selected from H, alkyl, alkenyl, haloalkyl, cycloalkyl, cycloalkylalkyl, aryl, aralkyl, aralkenyl, heterocyclyl or heterocyclylalkyl.

Of these compounds of formula (I), the preferred compounds are compounds of formula (I) having the following formula (Ia):

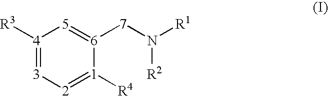

as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;

wherein:

R¹, R², R³ and R⁴ are selected as set forth in the following table for each compound of formula (Ia):

| Com- | | | | |
|---|---|---|---|---|
| pound | | | | |
| of | | | | |
| Formula | | | | |
| (Ia) | R¹ | R² | R³ | R⁴ |
| 1 | -Cyclopentyl | —H | —CF₃ | —H |
| 2 | -Isopropyl | -Isopropyl | —CF₃ | —H |
| 3 | -Furan-2-ylmethyl | —H | —CF₃ | —H |
| 4 | -Phenyl | —H | —CF₃ | —H |
| 5 | -Isobutyl | —H | —CF₃ | —H |
| 6 | -Cyclopropylmethyl | —H | —CF₃ | —H |
| 7 | -Benzyl | —H | —CF₃ | —H |
| 8 | -Cyclohexylmethyl | —H | —CF₃ | —H |
| 9 | -Benzyl | -Methyl | —CF₃ | —H |
| 10 | -Tetrahydrofuran-2- | —H | —CF₃ | —H |
| | ylmethyl | | | |
| 11 | —CH₂CH₂OH | —H | —CF₃ | —H |
| 12 | -3-Trifluoromethylbenzyl | —H | —CF₃ | —H |
| 13 | —CH₂CH₂OH | —CH₂CH₂OH | —CF₃ | —H |
| 14 | —CH₂CH₂NH₂ | —H | —CF₃ | —H |
| 15 | -Methyl | —H | —CF₃ | —Cl |
| 16 | -Propyl | —H | —CF₃ | —Cl |
| 17 | —CH₂CH₂OH | —H | —CF₃ | —Cl |
| 18 | -Tetrahydrofuran-2- | —H | —CF₃ | —Cl |
| | ylmethyl | | | |
| 19 | -Cyclohexyl | —H | —CF₃ | —Cl |
| 20 | -Methyl | -Methyl | —CF₃ | —Cl |
| 21 | —CH₂CH₂Cl | —H | —CF₃ | —Cl |
| 22 | —CH₂CH₂NH₂ | —H | —CF₃ | —Cl |
| 23 | -tert-Butyl | —H | —CF₃ | —Cl |
| 24 | -tert-Butyl | —H | —CF₃ | —H |
| 25 | -(2-Chloro-5- | —H | —CF₃ | —Cl |
| | trifluoromethyl)benzyl | | | |
| 26 | —CH₂CH₂CH₂NH₂ | —H | —CF₃ | —Cl |
| 27 | —(CH₂)₅— | | —CF₃ | —Cl |
| 28 | —(CH₂)₄— | | —CF₃ | —Cl |
| 29 | —(CH₂)₂—O—(CH₂)₂— | | —CF₃ | —Cl |
| 30 | -Ethyl | -Ethyl | —CF₃ | —Cl |
| 31 | -(2-1H-Indol-3-yl)ethyl | —H | —CF₃ | —Cl |
| 32 | -Furan-2-ylmethyl | —H | —CF₃ | —Cl |
| 33 | -Benzyl | —H | —CF₃ | —Cl |
| 34 | -Isobutyl | —H | —CF₃ | —Cl |
| 35 | -(1H-Imidazol-2-yl)methyl | —H | —CF₃ | —H |
| 36 | -Decahydronaphthalen-2-yl | —H | —CF₃ | —H |
| 37 | —H | —H | —CF₃ | -Methyl |

and for each compound of formula (Ia) as set forth above:

the numerals 1 through 7 in formula (Ia) represent carbon atoms;

the carbon atoms at numerals 2, 3 and 5 are independently optionally substituted with —X, —R or —OR;

the carbon atom at numeral 7 is independently substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;

each X is independently selected from fluoro, bromo, chloro or iodo; and

each R is independently selected from H, alkyl, alkenyl, haloalkyl, cycloalkyl, cycloalkylalkyl, aryl, aralkyl, aralkenyl, heterocyclyl or heterocyclylalkyl.

Of these compounds, the most preferred compounds are those compounds where the carbon atoms at numerals 2, 3 and 5 are substituted with H; and the carbon atom at numeral 7 is substituted with two H's.

### C. PHARMACEUTICAL COMPOSITIONS OF THE INVENTION

The present invention provides a pharmaceutical or veterinary composition (hereinafter, collectively referred to as a pharmaceutical composition) containing a compound of the invention as described above, in admixture with a pharmaceutically acceptable carrier. The invention further provides a composition, preferably a pharmaceutical composition, containing an effective amount of a compound as described above, in association with a pharmaceutically acceptable carrier.

The pharmaceutical compositions of the present invention may be in any form which allows for the composition to be administered to a patient. For example, the composition may be in the form of a solid, liquid or gas (aerosol). Typical routes of administration include, without limitation, oral, topical, parenteral, sublingual, rectal, vaginal, ocular, and intranasal. The term parenteral as used herein includes subcutaneous injections, intravenous, intramuscular, intrasternal injection or infusion techniques. Pharmaceutical composition of the invention are formulated so as to allow the active ingredients contained therein to be bioavailable upon administration of the composition to a patient. Compositions that will be administered to a patient take the form of one or more dosage units, where for example, a tablet may be a single dosage unit, and a container of a compound of the invention in aerosol form may hold a plurality of dosage units.

Materials used in preparing the pharmaceutical compositions should be pharmaceutically pure and non-toxic in the amounts used. It will be evident to those of ordinary skill in the art that the optimal dosage of the active ingredient(s) in the pharmaceutical composition will depend on a variety of factors. Relevant factors include, without limitation, the type of subject (e.g., human), the particular form of the active ingredient, the manner of administration and the composition employed.

In general, the pharmaceutical composition includes an (where “a” and “an” refers here, and throughout this specification, as one or more) active compound of the invention as described herein, in admixture with one or more carriers. The carrier(s) may be particulate, so that the compositions are, for example, in tablet or powder form. The carrier(s) may be liquid, with the compositions being, for example, an oral syrup or injectable liquid. In addition, the carrier(s) may be gaseous, so as to provide an aerosol composition useful in, e.g., inhalatory administration.

When intended for oral administration, the composition is preferably in either solid or liquid form, where semi-solid, semi-liquid, suspension and gel forms are included within the forms considered herein as either solid or liquid.

As a solid composition for oral administration, the composition may be formulated into a powder, granule, compressed tablet, pill, capsule, chewing gum, wafer or the like form. Such a solid composition will typically contain one or more inert diluents or edible carriers. In addition, one or more of the following adjuvants may be present: binders such as carboxymethylcellulose, ethyl cellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the composition is in the form of a capsule, e.g., a gelatin capsule, it may contain, in addition to materials of the above type, a liquid carrier such as polyethylene glycol, cyclodextrin or a fatty oil.

The composition may be in the form of a liquid, e.g., an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid composition intended for either parenteral or oral administration should contain an amount of a compound of the invention such that a suitable dosage will be obtained. Typically, this amount is at least 0.01% of a compound of the invention in the composition. When intended for oral administration, this amount may be varied to be between 0.1% and about 80% of the weight of the composition. Preferred oral compositions contain between about 4% and about 50% of the active compound of the invention. Preferred compositions and preparations according to the present invention are prepared so that a parenteral dosage unit contains between 0.01% to 2% by weight of active compound.

The pharmaceutical composition may be intended for topical administration, in which case the carrier may suitably comprise a solution, emulsion, ointment or gel base. The base, for example, may comprise one or more of the following: petrolatum, lanolin, polyethylene glycols, beeswax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device. Topical formulations may contain a concentration of the compound of formula the invention of from about 0.01% to about 10% w/v (weight per unit volume).

The composition may be intended for rectal administration, in the form, e.g., of a suppository which will melt in the rectum and release the drug. The composition for rectal administration may contain an oleaginous base as a suitable nonirritating excipient. Such bases include, without limitation, lanolin, cocoa butter and polyethylene glycol.

The composition may include various materials which modify the physical form of a solid or liquid dosage unit. For example, the composition may include materials that form a coating shell around the active ingredients. The materials which form the coating shell are typically inert, and may be selected from, for example, sugar, shellac, and other enteric coating agents. Alternatively, the active ingredients may be encased in a gelatin capsule.

The composition in solid or liquid form may include an agent which binds to the active component(s) and thereby assists in the delivery of the active components. Suitable agents which may act in this capacity include a monoclonal or polyclonal antibody, a protein or a liposome.

The pharmaceutical composition of the present invention may consist of gaseous dosage units, e.g., it may be in the form of an aerosol. The term aerosol is used to denote a variety of systems ranging from those of colloidal nature to systems consisting of pressurized packages. Delivery may be by a liquefied or compressed gas or by a suitable pump system which dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, spacers and the like, which together may form a kit. Preferred aerosols may be determined by one skilled in the art, without undue experimentation.

Whether in solid, liquid or gaseous form, the pharmaceutical composition of the present invention may contain one or more known pharmacological agents used in the treatment of nicotine dependence.

The pharmaceutical compositions may be prepared by methodology well known in the pharmaceutical art.

A pharmaceutical composition intended to be administered by injection can be prepared by combining the compound of the invention with water so as to form a solution. A surfactant may be added to facilitate the formation of a homogeneous solution or suspension. Surfactants are compounds that non-covalently interact with the compound of the invention so as to facilitate dissolution or homogeneous suspension of the active compound in the aqueous delivery system.

### D. UTILITY OF THE COMPOUNDS AND PHARMACEUTICAL COMPOSITIONS OF THE INVENTION

The compounds and compositions of the invention are CYP2A inhibitors and are therefore useful in treating nicotine dependence in a subject.

One embodiment of the invention is a method for inhibiting the activity of an isoform of a CYP2A enzyme comprising administering to a subject a therapeutically effective amount of a compound or a composition of the invention.

Of this embodiment, a further embodiment is where the CYP2A isoform is CYP2A6.

Of this embodiment, a further embodiment is where the effect of inhibition of the CYP2A enzyme activity is an increase in the circulating plasma half-life of nicotine in the subject.

Another embodiment of the invention is a method of treating nicotine dependence in a subject resulting from the use of tobacco containing products, wherein the method comprises administering to the subject a therapeutically effective amount of a compound or a composition of the invention.

Of this embodiment, a further embodiment is where the nicotine dependence is the result of cigarette smoking.

Of this embodiment, a further embodiment is where the method results in a reduction of the number of cigarettes smoked by the subject over a period of time.

Of this embodiment, a further embodiment is where the method results in a cessation of cigarette smoking by the subject.

Another embodiment of the invention is a method of treating nicotine dependence in a subject resulting from the use of tobacco containing products,

wherein the method comprises co-administering to a subject a therapeutically effective amount of a compound or a composition of the invention and a pharmacological smoking reduction therapy.

Of this embodiment, a further embodiment is where the nicotine dependence is the result of cigarette smoking.

Of this embodiment, a further embodiment is where the method results in a reduction in the number of cigarettes smoked by the subject over a period of time.

Of this embodiment, a further embodiment is where the method results in a cessation of cigarette smoking by the subject.

Another embodiment of the invention is a method of treating nicotine dependence in a subject resulting from the use of tobacco containing products, wherein the method comprises administering to the subject a therapeutically effective amount of a compound or composition of the invention and administering behavioral or alternative smoking reduction therapy to the subject.

Of this embodiment, a further embodiment is where the nicotine dependence is the result of cigarette smoking.

Of this embodiment, a further embodiment is where the method results in a reduction in the number of cigarettes smoked by the subject over a period of time.

Of this embodiment, a further embodiment is where the objective or result is cessation of cigarette smoking.

Another embodiment of the invention is a method of reducing exposure of a subject to certain compounds comprising administering a therapeutically effective amount of a compound or composition of the invention.

Of this embodiment, a further embodiment is where the exposure to the compounds results from use of tobacco products.

Of this embodiment, a further embodiment is where the compounds are carcinogens or other physiologically harmful compounds.

Of this embodiment, a further embodiment is where the method results in a reduction in the amount of tobacco product used by the subject.

Of this embodiment, a further embodiment is where the method results in a cessation of use of tobacco products by the subject.

Of this embodiment, a further embodiment is where the compounds are procarcinogens or precursors to carcinogens or other physiologically harmful compounds.

Of this embodiment, a further embodiment is wherein a conversion of the procarginogen or the precursor or otherwise harmful compound is inhibited.

### E. PREPARATION OF COMPOUNDS OF THE INVENTION

The compounds of the invention can be prepared by methods employing steps known to those skilled in the art or analogous to those steps. General synthetic methods can be found in “Comprehensive Organic Transformations”, R. C. Larock, VCH Publishers, New York, N.Y., 1989 and references cited therein. Additional literature references useful for the synthesis of compounds of the invention are as follows:

In particular, compounds of the invention may be prepared by the following Reaction Schemes or by the following Synthetic Examples. It is understood that other compounds of the invention may be prepared in a similar manner as described below or by methods known to one of ordinary skill in the art.

In general, in the following Reaction Scheme 1, nucleophilic displacement of X in a compound such as 1 gives compounds such as 2 having a R⁴ substituents. R⁴ substituents may undergo various modifications known to those skilled in the art to generate other R⁴ groups. Reduction of the nitrile group in 2 using, for example, borane or catalytic hydrogenation, gives the benzyl amine compounds of 3. The benzylic amino group in 3 may undergo various modifications known to those skilled in the art to introduce numerous R¹ and R² groups in compounds such as 4.

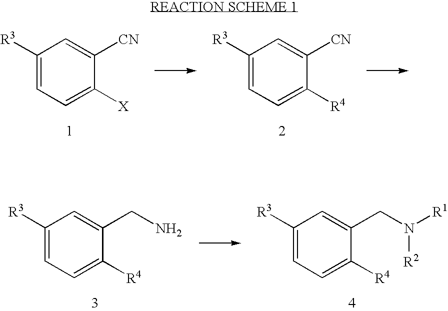

### Synthetic Example 1.0

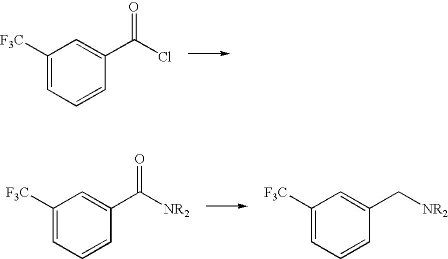

1.1 Cyclopentyl-(3-(trifluoromethyl)benzyl)amine, hydrochloride salt:

A solution of 3-(trifluoromethyl)benzoyl chloride (0.30 ml, 2.0 mmol) in CH₂Cl₂ (3 ml) was added to an ice cooled solution of cyclopentylamine (0.20 ml, 2.0 mmol), Et₃N (0.29 ml, 2.0 mmol) and CH₂Cl₂ (2 ml). The resulting solution was allowed to stir at room temperature for 3 hours then was concentrated. The residue was triturated with EtOAc and was filtered. The filtrate was concentrated to give N-cyclopentyl-3-(trifluoromethyl)benzamide. LiAlH₄ (2.0 ml of a 1 M solution in THF) was added to a solution of the crude amide (2.0 mmol) in THF (5 ml) and the resulting solution was heated at reflux overnight. ¹H NMR analysis indicated the reaction was about 70% complete therefore another 2 ml of a 1 M solution of LiAlH₄ in THF was added and the reaction was allowed to continue at reflux for 5 hours. The reaction was quenched with Na₂SO₄.10H₂O then was filtered and concentrated. Purification using column chromatography on silica gel, eluting with 25% EtOAc/hexanes then 10% MeOH/EtOAc afforded 260 mg (46%) of cyclopentyl-(3-(trifluoromethyl)benzyl)amine, hydrochloride salt.

The following benzylamines were prepared using the procedure described in 1.1. In all examples 2.0 mmol of 3-(trifluoromethyl)benzoyl chloride was reacted with 2.0 mmol of a primary or secondary amine to give an amide intermediate. The amide intermediates were reduced with 5.0 mmol of LiAlH₄ to give amines that were treated with concentrated HCl solution to form the salts.

1.2 Diisopropyl-(3-trifluoromethylbenzyl)amine, hydrochloride salt (370 mg, 62%).

1.3 Propyl-(3-trifluoromethylbenzyl)amine, hydrochloride salt (343 mg, 68%).

1.4 1-(3-trifluoromethylbenzyl)pyrrolidine, hydrochloride salt (335 mg, 68%).

1.5 Furan-2-ylmethyl-(3-trifluoromethylbenzyl)amine, hydrochloride salt (495 mg, 85%).

1.6 Phenyl-(3-trifluoromethylbenzyl)amine, hydrogen chloride salt (535 mg, 93%).

1.7 Isobutyl-(3-trifluoromethylbenzyl)amine, hydrogen chloride salt (138 mg, 26%) was obtained after further purification using ion exchange chromatography, eluting with 2M NH₃ in MeOH followed by treatment with concentrated HCl.

### Synthetic Example 2.0

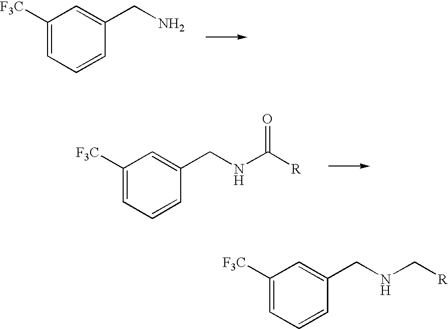

The following benzylamines were prepared using the procedure described above. In all examples, equimolar quantities of 3-(trifluoromethyl)benzylamine and acid chlorides was reacted to give an amide intermediate. The amide intermediates were reduced with 2.5 molar equivalents of LiAlH₄ to give amines that were treated with concentrated HCl solution to form the salts.

2.1 Cyclopropylmethyl-(3-trifluoromethylbenzyl)amine, hydrogen chloride salt (470 mg, 88%) was obtained pure by using ion exchange chromatography, eluting with 2M NH₃ in MeOH prior to treatment with HCl.

2.2 Benzyl-(3-trifluoromethylbenzyl)amine, hydrogen chloride salt (217 mg, 36%) was obtained pure by using ion exchange chromatography, eluting with 2M NH₃ in MeOH prior to treatment with HCl.

2.3 Butyl-(3-trifluoromethylbenzyl)amine, hydrogen chloride salt (290 mg, 54%) was obtained pure by using ion exchange chromatography, eluting with 2M NH₃ in MeOH prior to treatment with HCl.

2.4 Ethyl-(3-trifluoromethylbenzyl)amine, hydrogen chloride salt (30 mg, 20%) was obtained without chromatographic purification.

2.5 Cyclohexylmethyl-(3-trifluoromethylbenzyl)amine, hydrochloride salt (160 mg, 55%).

### Synthetic Example 3.0

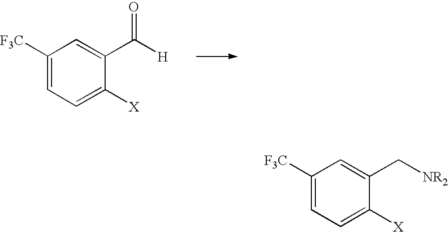

3.1 4-(3-Trifluoromethylbenzyl)morpholine, hydrochloride salt.

A solution of 3-trifluoromethylbenzaldehyde (1.5 mmol), morpholine (1.0 mmol), MP-CNBH₃ (3.0 mmol) and MeOH (5 ml) was stirred at room temperature overnight. The reaction mixture was filtered and concentrated. The residue was purified using ion exchange chromatography, eluting with 2M NH₃ in MeOH. The amine was reacted with concentrated HCl followed by co-evaporation with EtOAc to afford 4-(3-trifluoromethylbenzyl)morpholine, hydrochloride salt (50 mg, 18%).

3.2 1-(3-Trifluoromethylbenzyl)piperidine, hydrochloride salt

The procedure described in 3.1, except substitution by piperidine and further purification of the amine using column chromatography on silica gel, eluting with EtOAc/MeOH/H₂O/Et₃N (9:1:0.2:0.2) prior to treatment with HCl, afforded 1-(3-trifluoromethylbenzyl)piperidine, hydrochloride salt (28 mg, 10%).

3.3 Benzyl-methyl-(3-trifluoromethylbenzyl)-amine, hydrochloride salt.

The procedure described in 3.2, except substitution by benzyl-methyl-amine, afforded benzyl-methyl-(3-trifluoromethylbenzyl)-amine, hydrochloride salt (46 mg, 15%).

3.4 Diethyl-(3-trifluoromethylbenzyl)-amine, hydrochloride salt.

The procedure described in 3.2, except substitution by diethylamine, afforded diethyl-(3-trifluoromethylbenzyl)-amine, hydrochloride salt (6 mg, 2%).

3.5 (Tetrahydrofuran-2-ylmethyl)-(3-trifluoromethylbenzyl)-amine, hydrochloride salt.

A solution of 3-trifluoromethylbenzaldehyde (1.5 mmol), tetrahydrofurfurylamine (1.0 mmol), trimethylorthoformate (4 ml) and MeOH (2 ml) was stirred at room temperature. After 3 hours, MP-BH₄ (3 mmol) was added and the reaction was allowed to continue overnight. The solution was filtered and concentrated and the residue was purified by column chromatography on silica gel eluting with EtOAc/MeOH/H₂O/Et₃N (9:1:0.2:0.2). Treatment with HCl afforded (tetrahydrofuran-2-ylmethyl)-(3-trifluoromethylbenzyl)-amine, hydrochloride salt (109 mg, 37%).

3.6 [2-(1H-lndol-3-yl)ethyl]-(3-trifluoromethylbenzyl)-amine, hydrochloride salt.

The procedure described in 3.5, except substitution by 2-(1H-indol-3-yl)ethylamine, afforded [2-(1H-indol-3-yl)ethyl]-(3-trifluoromethylbenzyl)-amine, hydrochloride salt (122 mg, 34%).

3.7 2-(3-Trifluoromethylbenzylamino)ethanol, hydrochloride salt.

The procedure described in 3.5, except substitution by 2-aminoethanol and further purification using ion-exchange chromatography eluting with 2M NH₃ in MeOH prior to salt formation, afforded 2-(3-trifluoromethylbenzylamino)ethanol, hydrochloride salt (110 mg, 43%).

3.8 Pentyl-(3-trifluoromethylbenzyl)-amine, hydrochloride salt.

The procedure described in 3.7, except substitution by pentylamine, afforded pentyl-(3-trifluoromethylbenzyl)-amine, hydrochloride salt (133 mg, 47%).

3.9 Pentyl-(3-trifluoromethylbenzyl)-amine, hydrochloride salt.

The procedure described in 3.7, except substitution by pentylamine, afforded pentyl-(3-trifluoromethylbenzyl)-amine, hydrochloride salt (133 mg, 47%).

3.10 Bis-(3-trifluoromethylbenzyl)-amine, hydrochloride salt.

The procedure described in 3.7, except substitution by 3-trifluoromethylbenzylamine, afforded bis-(3-trifluoromethylbenzyl)-amine, hydrochloride salt (130 mg, 35%).

3.11 Bis-(2-hydroxyethyl)-(3-trifluoromethylbenzyl)-amine, hydrochloride salt.

A solution of 3-trifluoromethylbenzaldehyde (0.75 mmol), diethanolamine (0.50 mmol), THF (3 ml) and MP-B(OAc)₃H (1 g) was stirred at room temperature overnight. The solution was filtered and concentrated and the residue was purified by column chromatography on silica gel. The free amine was dissolved in MeOH and treated with concentrated HCl (100 μl) then was concentrated to afford bis-(2-hydroxyethyl)-(3-trifluoromethylbenzyl)-amine, hydrochloride salt (22 mg, 15%) as a pale yellow waxy solid.

3.12 Dimethyl-(3-trifluoromethylbenzyl)-amine, hydrochloride salt.

A solution of 3-trifluoromethylbenzaldehyde (0.75 mmol), dimethylamine hydrochloride (0.50 mmol), DMF (4 ml) and MP-B(OAc)₃H (1.5 g) was stirred at room temperature overnight. The solution was filtered and the filtrate was stirred with MP-TsOH (1.0 mmol) for 1 hour. The resin was filtered and washed with CH₂Cl₂ and MeOH. Eluting with 2M NH₃ in MeOH followed by concentration in vacuo afforded the free amine. The free amine was dissolved in MeOH and treated with concentrated HCl (100 μl) then was concentrated to afford dimethyl-(3-trifluoromethylbenzyl)-amine, hydrochloride salt (24 mg, 20%) as a pale yellow solid.

3.13 Ethylamine-(3-trifluoromethylbenzyl)-amine, dihydrochloride salt.

A solution of 3-trifluoromethylbenzaldehyde (174 mg, 1.0 mmol), ethylenediamine dihydrochloride (200 mg, 1.5 mmol), Et₃N (3.0 mmol), MeOH (3 ml) and trimethyl orthoformate (3 ml) was stirred at room temperature for 3 hours. To this solution was added MP-BH₄ (1.0 g, 3.3 mmol) and the reaction was allowed to continue for 3 days. The solution was filtered, washing with MeOH, then was concentrated. The residue was dissolved in CH₂Cl₂ (75 ml) and MeOH (5 ml) and was washed with saturated NaHCO₃ solution (30 ml) and water (2×30 ml). The concentrated organic layer was purified by column chromatography on silica gel eluting with 0.2 M NH₃ in MeOH. The free amine was dissolved in MeOH and treated with concentrated HCl (150 μl) then was concentrated to afford ethylamine-(3-trifluoromethylbenzyl)-amine, dihydrochloride salt (57 mg, 20%).

3.14 (2-Chloro-5-trifluoromethylbenzyl)-methyl-amine, hydrochloride salt.

A solution of 2-chloro-5-trifluoromethylbenzaldehyde (0.75 mmol), 2 mM methylamine in THF (0.50 mmol), MeOH (1 ml) and trimethyl orthoformate (3 ml) was stirred at room temperature for 3 hours. To this solution was added MP-BH₄ (2.25 mmol) and the reaction was allowed to continue overnight. The reaction mixture was filtered and concentrated. The residue was purified using ion exchange chromatography eluting with 2 M NH₃ in MeOH. The free amine was dissolved in MeOH and treated with concentrated HCl (200 μl) and concentrated to afford (2-Chloro-5-trifluoromethylbenzyl)-methyl-amine, hydrochloride salt (81 mg, 62%).

3.15 Propyl-(2-chloro-5-trifluoromethylbenzyl)-amine, hydrochloride salt.

The procedure described in 3.14, except substitution by propylamine, afforded propyl-(2-chloro-5-trifluoromethylbenzyl)-amine, hydrochloride salt (63 mg, 44%).

3.16 2-(2-Chloro-5-trifluoromethylbenzylamino)-ethanol, hydrochloride salt.

The procedure described in 3.14, except substitution by ethanolamine, afforded 2-(2-chloro-5-trifluoromethylbenzylamino)-ethanol, hydrochloride salt (110 mg, 76%).

3.17 (2-Chloro-5-trifluoromethylbenzyl)-(tetrahydrofuran-2-ylmethyl)-amine, hydrochloride salt.

3.18 The procedure described in 3.14, except substitution by tetrahydrofuran-2-ylmethylamine, afforded (2-chloro-5-trifluoromethylbenzyl)-(tetrahydrofuran-2-ylmethyl)-amine, hydrochloride salt (120 mg, 73%).

3.19 (2-Chloro-5-trifluoromethylbenzyl)-cyclohexyl-amine, hydrochloride salt.

The procedure described in 3.14, except substitution by tetrahydrofuran-2-ylmethylamine, afforded (2-chloro-5-trifluoromethylbenzyl)-cyclohexyl-amine, hydrochloride salt (120 mg, 72%).

3.20 (2-Chloro-5-trifluoromethylbenzyl)-dimethyl-amine, hydrochloride salt.

A solution of 2-chloro-5-trifluoromethylbenzaldehyde (0.75 mmol), dimethylamine hydrochloride (0.75 mmol), DMF (5 ml) and MP-B( )Ac)3H was allowed to react at room temperature overnight. The reaction mixture was filtered and the filtrate was stirred with MP-TsOH (1.0 g) for 1 hour. The resin was filtered and washed with CH₂Cl₂ and MeOH. Eluting with 2M NH₃ in MeOH followed by concentration in vacuo afforded the free amine. The free amine was dissolved in MeOH and treated with concentrated HCl (100 μl) then was concentrated to afford (2-chloro-5-trifluoromethylbenzyl)-dimethyl-amine, hydrochloride salt (61 mg, 36%).

3.21 (2-Chloroethyl)-(2-Chloro-5-trifluoromethylbenzyl)-amine, hydrochloride salt.

The procedure described in 3.20, except substitution by 2-chloroethylamine hydrochloride, afforded (2-chloroethyl)-(2-chloro-5-trifluoromethylbenzyl)-amine, hydrochloride salt (93 mg, 40%).

3.22 Ethylamine-(2-chloro-5-trifluoromethylbenzyl)-amine, dihydrochloride salt.

The procedure described in 3.13 afforded ethylamine-(2-chloro-5-trifluoromethylbenzyl)-amine, dihydrochloride salt (52 mg, 16%).

3.23 tert-Butyl-(2-chloro-5-trifluoromethylbenzyl)-amine, hydrochloride salt.

The procedure described in 3.14, except substitution by tert-butylamine, afforded tert-butyl-(2-chloro-5-trifluoromethylbenzyl)-amine, hydrochloride salt (30 mg, 20%).

3.24 tert-Butyl-(3-trifluoromethylbenzyl)-amine, hydrochloride salt.

The procedure described in 3.23, except substitution by 3-trifluoromethybenzaldehyde, afforded tert-butyl-(3-trifluoromethylbenzyl)-amine, hydrochloride salt (33 mg, 25%).

3.25 Bis-(2-chloro-5-trifluoromethylbenzyl)-amine, hydrochloride salt.

The procedure described in 3.14, except substitution by 2-chloro-5-trifluoromethylbenzylamine, afforded bis-(2-chloro-5-trifluoromethylbenzyl)-amine, hydrochloride salt (80 mg, 37%).

3.26 Ethylamino-(2-chloro-5-trifluoromethylbenzyl)-amine, dihydrochloride salt.

A solution of 2-chloro-5-trifluoromethylbenzaldehyde (1 mmol), ethylenediamine dihydrochloride (2 mmol), MeOH (3 ml) and trimethyl orthoformate (3 ml) was stirred at room temperature for 3 hours. To this solution was added MP-BH₄ (2 g) and the reaction was allowed to continue overnight. The reaction mixture was filtered and concentrated. The residue was purified by column chromatography on-silica gel eluting with EtOAc/MeOH/H₂O/Et₃N (7:2:0.5:0.5). The free amine was dissolved in MeOH and treated with concentrated HCl to afford ethylamino-(2-chloro-5-trifluoromethylbenzyl)-amine, dihydrochloride salt (64 mg, 19%).

3.27 3-Propylamino-(3-trifluoromethylbenzyl)-amine, dihydrochloride salt.

The procedure described in 3.26, except substitution by 1,3-diaminopropane, afforded 3-propylamino-(3-trifluoromethylbenzyl)-amine, dihydrochloride salt (66 mg, 22%).

3.28 1-(2-Chloro-5-trifluoromethylbenzyl)-piperidine, hydrochloride salt.

A solution of 2-chloro-5-trifluoromethylbenzaldehyde (0.75 mmol), piperidine (0.5 mmol), THF (5 ml) and MP-B(OAC)₃H (1.5 mmol) was stirred at room temperature overnight. The solution was filtered and concentrated and the residue was purified first by ion-exchange chromatography eluting with 2M NH₃ in MeOH followed by column chromatography on silica gel. The free amine was dissolved in MeOH and treated with 6M HCl then was concentrated to afford 1-(2-chloro-5-trifluoromethylbenzyl)-piperidine, hydrochloride salt (72 mg, 46%).

3.29 1-(2-Chloro-5-trifluoromethylbenzyl)-pyrrolidine, hydrochloride salt.

The procedure described in 3.28, except substitution by pyrrolidine, afforded 1-(2-chloro-5-trifluoromethylbenzyl)-pyrrolidine, hydrochloride salt (103 mg, 69%).

3.30 4-(2-Chloro-5-trifluoromethylbenzyl)-morpholine, hydrochloride salt.

The procedure described in 3.28, except substitution by morpholine, afforded 1-(2-chloro-5-trifluoromethylbenzyl)-morpholine, hydrochloride salt (18 mg, 11%). 3.31 (2-Chloro-5-trifluoromethylbenzyl)-diethyl-amine, hydrochloride salt.

The procedure described in 3.28, except substitution by diethylamine, afforded 1-(2-chloro-5-trifluoromethylbenzyl)-diethylamine, hydrochloride salt (3 mg, 2%).

3.32 (2-Chloro-5-trifluoromethylbenzyl)-[2-(1H-indol-3-yl)-ethyl]-amine, hydrochloride salt.

A solution of 2-chloro-5-trifluoromethylbenzaldehyde (173 mg, 0.825 mmol), tryptamine (88 mg, 0.55 mmol), trimethyl orthoformate (3 ml) and MeOH (3 ml) was stirred at room temperature for 3 hours. To this solution was added MP-BH₄ (1.2 g, 3.7 mmol) and the reaction was allowed to continue overnight. The solution was filtered and concentrated then the residue was purified by ion exchange chromatography eluting with 2M NH₃ in MeOH. The free amine was dissolved in MeOH and treated with HCl to afford (2-chloro-5-trifluoromethylbenzyl)-[2-(1H-indol-3-yl)-ethyl]-amine, hydrochloride salt (203 mg, 87%).

3.33 2-Chloro-5-trifluoromethylbenzyl)-furan-2-ylmethyl-amine, hydrochloride salt.

A solution of 2-chloro-5-trifluoromethylbenzylamine (0.50 mmol), 2-furaldehyde

(0.75 mmol), trimethyl orthoformate (2 ml) and MeOH (2.5 ml) was stirred at room temperature for 3 hours. To this solution was added MP-BH₄ (1.5 mmol) and the reaction was allowed to continue overnight. The reaction mixture was filtered and concentrated then the residue was purified using ion exchange chromatography eluting with 2M NH₃ MeOH. The free amine was dissolved in MeOH and was treated with concentrated HCl to afford 2-chloro-5-trifluoromethylbenzyl)-furan-2-ylmethyl-amine, hydrochloride salt (94 mg, 58%).

3.34 Benzyl-(2-chloro-5-trifluoromethylbenzyl)-amine, hydrochloride salt.

The procedure described in 3.33, except substitution by benzaldehyde, afforded benzyl-(2-chloro-5-trifluoromethylbenzyl)-amine, hydrochloride salt (116 mg, 69%).

3.35 2-Chloro-5-trifluoromethylbenzyl)-isobutyl-amine, hydrochloride salt.

The procedure described in 3.33, except substitution by isobutyraldehyde and additional purification by column chromatography on silica gel eluting with 10% MeOH/EtOAc, afforded benzyl-(2-chloro-5-trifluoromethylbenzyl)-amine, hydrochloride salt (44 mg, 29%).

3.36 (1H-Imidazol-2-ylmethyl)-(3-trifluoromethylbenzyl)-amine, dihydrochloride salt.

The procedure described in 3.33, except substitution by 2-imidazolecarboxaldehyde, afforded (1H-imidazol-2-ylmethyl)-(3-trifluoromethylbenzyl)-amine, dihydrochloride salt (164 mg, 100%). 3.37 Decahydronaphthalen-2-yl)-(3-trifluoromethylbenzyl)-amine, hydrochloride salt.

A solution of 3-trifluoromethylbenzylamine (1.0 mmol), decahydro-2-naphthalenone (1.5 mmol), trimethyl orthoformate (4 ml) and MeOH (5 ml) was stirred at room temperature for 4 hours. To this solution was added MP-(CN)BH₃ (3.0 mmol) and the reaction was allowed to continue for 24 hours. The reaction mixture was filtered and concentrated. The free amine was treated with concentrated HCl and the resulting salt was purified by column chromatography eluting with 10% MeOH/EtOAc to afford decahydronaphthalen-2-yl)-(3-trifluoromethylbenzyl)-amine, hydrochloride salt (23 mg, 7%).

### Synthetic Example 4.0

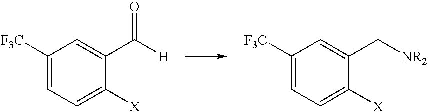

4.1 2-Methyl-5-trifluoromethylbenzylamine, hydrochloride salt.

Lithium aluminum hydride (3.2 ml of a 1 M solution in Et₂O) was added to an ice-cold solution of 2-methyl-5-trifluoromethylbenzonitrile (Indofine) (298 mg, 1.61 mmol) in Et₂O (20 ml) under argon. After 15 minutes the cold bath was removed and the reaction was allowed to continue at room temperature for 3 hours. The reaction mixture was cooled in ice and was quenched by the addition of Na₂SO₄.10H₂O (1.1 g) then was vigorously stirred for 3.5 hours. The solution was filtered and concentrated then the resulting residue was purified by column chromatography on silica gel eluting with 10% MeOH/EtOAc. The free amine was dissolved in MeOH and treated with 6M HCl (1 ml) then was concentrated to afford 2-methyl-5-trifluoromethylbenzylamine, hydrochloride salt (298 mg, 82%) as a white solid.

### Biological Assay 1.0

### S9 Potency Screening Assay

The CYP2A6 inhibitory potency of compounds of the invention or their metabolites in S9 was assayed in the following manner. The compounds of the invention (50 μM) were incubated in human liver S9 for 30 min before adding nicotine (20 μM) and were allowed to incubate for another 60 min before stopping the reaction by adding 500 μL of acetonitrile. Incubation volume was 500 μL comprising of 40 mM Tris Buffer pH 7.4, 1 mM NADPH, 250 μM G6P, 250 μM UDPGA, 1 U 6GPDH and 1 mg of S9 protein (Xenotech). After stopping the reaction, proteins were precipitated and the supernatant was injected on LCMS with appropriate external standard. Cotinine formation (nicotine CYP2A6 product) was monitored to check for potency of the compounds of the invention in inhibiting its formation. Appropriate controls were used.

The compounds of the invention showed inhibition of CYP2A6 under the conditions of this assay of greater than 80%. The most potent of the compounds showed greater than 99% inhibition.

### Biological Assay 2.0

### CYP2A6 Supersomes Potency Screening Assay

This assay is intended to screen for CYP2A6 inhibitory potency of compounds of the invention or their CYP2A6 metabolites in CYP2A6 supersomes. The test compounds (5 μM) were incubated in CYP2A6 supersomes (BD Gentest) for 30 min before adding nicotine (20 μM) and were allowed to incubate for another 30 min before stopping the reaction by adding 500 μL of acetonitrile. Incubation volume was 500 μL comprising of 40 mM Tris Buffer pH 7.4, 2 mM NADP, 5 mM G6P, 3 mM MgCl₂, 1 U 6GPDH, 30 μL of human liver cytosol (Xenotech) and 20 μmol of CYP2A6 supersomes. After stopping the reaction, proteins were precipitated and the supernatant was injected on LCMS with appropriate external standard. Cotinine formation (nicotine CYP2A6 product) was monitored to check for potency of IPL compounds in inhibiting its formation. Appropriate controls were used.

The compounds of the invention showed inhibition of CYP2A6 in supersomes under the conditions of this assay of greater than 80%. The most potent of the compounds showed greater than 98% inhibition.

### Biological Assay 3.0

### Selectivity Screening Assay

This assay is intended to screen for selectivity of the compounds of the invention towards 5 major CYPs involved in drug metabolism. As a preliminary screen, all used CYPs were tested using 20 pmol of P450 content, 20CM of CYP marker (marker vary with CYP used, see table below) and 5 μM or 1 μM of a test compound of the invention, all preincubated for 30 min in supersomes and then incubated for another 30 min with the CYP marker(s). Incubation volume was 500 μL comprising of 40 mM Tris Buffer pH 7.4, 2 mM NADP, 5 mM G6P, 3 mM MgCl₂, 1 U 6GPDH, 30 μL of human liver cytosol (Xenotech) in case of CYP2A6 only and 20 μmol of the CYP supersomes (BD Gentest). The percentage of inhibition of the marker's metabolite formation was monitored. The five major CYP enzymes, markers used and their corresponding metabolites monitored are listed in Table 1 below. Appropriate controls were used.

TABLE 1

| | MARKER | MARKER |
|---|---|---|
| CYP 450 | SUBSTRATES | METABOLITES |
| 1A2 | Phenacetin | Acetoaminophen |
| 2A6 | Nicotine | Cotinine |
| 3A4 | Testosterone | 6B-HO-Testosterone |
| | Dextromethorphan | 3-Methoxymorphinan |
| 2C9 | Diclofenac | 4-OH-Diclofenac |
| 2C19 | Omeprazole | 5-OH-Omeprazole |
| 2D6 | Dextromethorphan | Dextrophan |

A number of the compounds of the invention were tested in this assay and were found to have a profound selectivity towards CYP2A6. Most of those tested had no significant effect on the other 5 CYPs tested but inhibited CYP2A6 effectively.

All of the U.S. patents, U.S. patent application publications, U.S. patent applications, foreign patents, foreign patent applications and non-patent publications referred to in this specification and/or listed in the Application Data Sheet are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for inhibiting the activity of an isoform of a CYP2A enzyme in a subject, wherein the method comprises administering to the subject a therapeutically effective amount of a compound of formula (I): 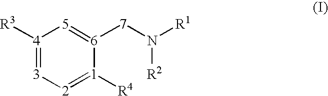
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹ and R² are independently selected from H, optionally substituted aryl, optionally substituted cycloalkyl, and optionally substituted C₁₋₃₀alkyl;
or R¹ and R², together with the nitrogen to which they are attached, form an optionally substituted heterocyclyl ring;
R³ is independently selected from X, optionally substituted C₁₋₃₀alkyl, C₁₋₃₀haloalkyl, nitro, carboxyl, —S(O)₂R, and —S(O)₂N(R)₂;
R⁴ is independently selected from H, X, optionally substituted C₁₋₃₀alkyl, C₁₋₃₀haloalkyl, —OR, —SR and —N(R)₂;
or R⁴ and R², together with the carbon and nitrogen to which they are attached, form an optionally substituted fused heterocyclyl ring;
where the numerals 1 through 7 in the formula (I) represent carbon atoms;
where the carbon atoms at numerals 2, 3 and 5 are independently optionally substituted with —X, —R, —N(R)₂ or —OR;
and where the carbon atom at numeral 7 is independently substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently fluoro, bromo, chloro or iodo; and
each R is independently selected from H and a C₁₋₃₀ organic moiety.

2. The method of claim 1 wherein the compound of formula (I) is a compound of formula (I) wherein:
R¹ and R² are independently selected from H; optionally substituted phenyl; optionally substituted benzyl; optionally substituted cyclopentyl; optionally substituted cyclohexyl; optionally substituted decahydronaphthalenyl; optionally substituted cyclopropylmethyl; optionally substituted cyclohexylmethyl; C₁₋₈alkyl optionally substituted with halo, —OR or —N(R)₂; optionally substituted furanylmethyl; optionally substituted tetrahydrofuranylmethyl; optionally substituted indolylethyl; and optionally substituted imidazolylmethyl;
or R¹ and R², together with the nitrogen to which they are attached, form an optionally substituted pyrrolidinyl, piperidinyl or morpholinyl ring;
R³ is independently selected from X, optionally substituted C₁₋₈alkyl, C₁₋₈haloalkyl, nitro, carboxyl, —S(O)₂R, and —S(O)₂N(R)₂;
R⁴ is independently selected from H, X, optionally substituted C₁₋₈alkyl, C₁₋₈haloalkyl, —OR, —SR and —N(R)₂;
the numerals 1 through 7 in formula (Ia) represent carbon atoms;
the carbon atoms at numerals 2, 3 and 5 are independently optionally substituted with —X, —R or —OR;
the carbon atom at numeral 7 is optionally substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

3. The method of claim 3 wherein the compound of formula (I) is a compound of formula (Ia): 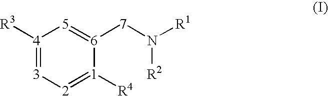
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹, R², R³ and R⁴ are selected as set forth in the following table for each compound of formula (Ia):

| Com- | | | | |
|---|---|---|---|---|
| pound | | | | |
| of | | | | |
| Formula | | | | |
| (Ia) | R¹ | R² | R³ | R⁴ |
| 1 | -Cyclopentyl | —H | —CF₃ | —H |
| 2 | -Isopropyl | -Isopropyl | —CF₃ | —H |
| 3 | -Furan-2-ylmethyl | —H | —CF₃ | —H |
| 4 | -Phenyl | —H | —CF₃ | —H |
| 5 | -Isobutyl | —H | —CF₃ | —H |
| 6 | -Cyclopropylmethyl | —H | —CF₃ | —H |
| 7 | -Benzyl | —H | —CF₃ | —H |
| 8 | -Cyclohexylmethyl | —H | —CF₃ | —H |
| 9 | -Benzyl | -Methyl | —CF₃ | —H |
| 10 | -Tetrahydrofuran-2- | —H | —CF₃ | —H |
| | ylmethyl | | | |
| 11 | —CH₂CH₂OH | —H | —CF₃ | —H |
| 12 | -3-Trifluoromethylbenzyl | —H | —CF₃ | —H |
| 13 | —CH₂CH₂OH | —CH₂CH₂OH | —CF₃ | —H |
| 14 | —CH₂CH₂NH₂ | —H | —CF₃ | —H |
| 15 | -Methyl | —H | —CF₃ | —Cl |
| 16 | -Propyl | —H | —CF₃ | —Cl |
| 17 | —CH₂CH₂OH | —H | —CF₃ | —Cl |
| 18 | -Tetrahydrofuran-2- | —H | —CF₃ | —Cl |
| | ylmethyl | | | |
| 19 | -Cyclohexyl | —H | —CF₃ | —Cl |
| 20 | -Methyl | -Methyl | —CF₃ | —Cl |
| 21 | —CH₂CH₂Cl | —H | —CF₃ | —Cl |
| 22 | —CH₂CH₂NH₂ | —H | —CF₃ | —Cl |
| 23 | -tert-Butyl | —H | —CF₃ | —Cl |
| 24 | -tert-Butyl | —H | —CF₃ | —H |
| 25 | -(2-Chloro-5- | —H | —CF₃ | —Cl |
| | trifluoromethyl)benzyl | | | |
| 26 | —CH₂CH₂CH₂NH₂ | —H | —CF₃ | —Cl |
| 27 | —(CH₂)₅— | | —CF₃ | —Cl |
| 28 | —(CH₂)₄— | | —CF₃ | —Cl |
| 29 | —(CH₂)₂—O—(CH₂)₂— | | —CF₃ | —Cl |
| 30 | -Ethyl | -Ethyl | —CF₃ | —Cl |
| 31 | -(2-1H-Indol-3-yl)ethyl | —H | —CF₃ | —Cl |
| 32 | -Furan-2-ylmethyl | —H | —CF₃ | —Cl |
| 33 | -Benzyl | —H | —CF₃ | —Cl |
| 34 | -Isobutyl | —H | —CF₃ | —Cl |
| 35 | -(1H-Imidazol-2-yl)methyl | —H | —CF₃ | —H |
| 36 | -Decahydronaphthalen-2-yl | —H | —CF₃ | —H |
| 37 | —H | —H | —CF₃ | -Methyl |

and for each compound of formula (Ia) as set forth above:
the numerals 1 through 7 in formula (Ia) represent carbon atoms;
the carbon atoms at numerals 2, 3 and 5 are independently optionally substituted with —X, —R or —OR;
the carbon atom at numeral 7 is independently substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

4. The method of claim 1 where the CYP2A isoform is CYP2A6.

5. The method of claim 4 where the effect of inhibition of the CYP2A enzyme activity is an increase in the circulating plasma half-life of nicotine in the subject.

6. A method of treating nicotine dependence in a subject resulting from the use of tobacco containing products, wherein the method comprises administering to the subject a therapeutically effective amount of a compound of formula (I): 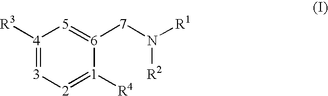
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹ and R² are independently selected from H, optionally substituted aryl, optionally substituted cycloalkyl, and optionally substituted C₁₋₃₀alkyl;
or R¹ and R², together with the nitrogen to which they are attached, form an optionally substituted heterocyclyl ring;
R³ is independently selected from X, optionally substituted C₁₋₃₀alkyl, C₁₋₃₀haloalkyl, nitro, carboxyl, —S(O)₂R, and —S(O)₂N(R)₂;
R⁴ is independently selected from H, X, optionally substituted C₁₋₃₀alkyl, C₁₋₃₀haloalkyl, —OR, —SR and —N(R)₂;
or R⁴ and R², together with the carbon and nitrogen to which they are attached, form an optionally substituted fused heterocyclyl ring;
where the numerals 1 through 7 in the formula (I) represent carbon atoms;
where the carbon atoms at numerals 2, 3 and 5 are independently optionally substituted with —X, —R, —N(R)₂ or —OR;
and where the carbon atom at numeral 7 is independently substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently fluoro, bromo, chloro or iodo; and
each R is independently selected from H and a C₁₋₃₀ organic moiety.

7. The method of claim 6 wherein the compound of formula (I) is a compound of formula (I) wherein:
R¹ and R² are independently selected from H; optionally substituted phenyl; optionally substituted benzyl; optionally substituted cyclopentyl; optionally substituted cyclohexyl; optionally substituted decahydronaphthalenyl; optionally substituted cyclopropylmethyl; optionally substituted cyclohexylmethyl; C₁₋₈alkyl optionally substituted with halo, —OR or —N(R)₂; optionally substituted furanylmethyl; optionally substituted tetrahydrofuranylmethyl; optionally substituted indolylethyl; and optionally substituted imidazolylmethyl;
or R¹ and R², together with the nitrogen to which they are attached, form an optionally substituted pyrrolidinyl, piperidinyl or morpholinyl ring;
R³ is independently selected from X, optionally substituted C₁₋₈alkyl, C₁₋₈haloalkyl, nitro, carboxyl, —S(O)₂R, and —S(O)₂N(R)₂;
R⁴ is independently selected from H, X, optionally substituted C₁₋₈alkyl, C₁₋₈haloalkyl, —OR, —SR and —N(R)₂;
the numerals 1 through 7 in formula (Ia) represent carbon atoms;
the carbon atoms at numerals 2, 3 and 5 are independently optionally substituted with —X, —R or —OR;
the carbon atom at numeral 7 is optionally substituted with:
a) one of the following: ═O, ═C(R)₂, ═C═C(R)₂, —C(R)₂[C(R)₂]ₙ— and —O[C(R)₂]O— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

8. The method of claim 7 wherein the compound of formula (I) is a compound of formula (Ia): 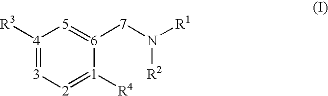
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹, R², R³ and R⁴ are selected as set forth in the following table for each compound of formula (Ia):

| Com- | | | | |
|---|---|---|---|---|
| pound | | | | |
| of | | | | |
| Formula | | | | |
| (Ia) | R¹ | R² | R³ | R⁴ |
| 1 | -Cyclopentyl | —H | —CF₃ | —H |
| 2 | -Isopropyl | -Isopropyl | —CF₃ | —H |
| 3 | -Furan-2-ylmethyl | —H | —CF₃ | —H |
| 4 | -Phenyl | —H | —CF₃ | —H |
| 5 | -Isobutyl | —H | —CF₃ | —H |
| 6 | -Cyclopropylmethyl | —H | —CF₃ | —H |
| 7 | -Benzyl | —H | —CF₃ | —H |
| 8 | -Cyclohexylmethyl | —H | —CF₃ | —H |
| 9 | -Benzyl | -Methyl | —CF₃ | —H |
| 10 | -Tetrahydrofuran-2- | —H | —CF₃ | —H |
| | ylmethyl | | | |
| 11 | —CH₂CH₂OH | —H | —CF₃ | —H |
| 12 | -3-Trifluoromethylbenzyl | —H | —CF₃ | —H |
| 13 | —CH₂CH₂OH | —CH₂CH₂OH | —CF₃ | —H |
| 14 | —CH₂CH₂NH₂ | —H | —CF₃ | —H |
| 15 | -Methyl | —H | —CF₃ | —Cl |
| 16 | -Propyl | —H | —CF₃ | —Cl |
| 17 | —CH₂CH₂OH | —H | —CF₃ | —Cl |
| 18 | -Tetrahydrofuran-2- | —H | —CF₃ | —Cl |
| | ylmethyl | | | |
| 19 | -Cyclohexyl | —H | —CF₃ | —Cl |
| 20 | -Methyl | -Methyl | —CF₃ | —Cl |
| 21 | —CH₂CH₂Cl | —H | —CF₃ | —Cl |
| 22 | —CH₂CH₂NH₂ | —H | —CF₃ | —Cl |
| 23 | -tert-Butyl | —H | —CF₃ | —Cl |
| 24 | -tert-Butyl | —H | —CF₃ | —H |
| 25 | -(2-Chloro-5- | —H | —CF₃ | —Cl |
| | trifluoromethyl)benzyl | | | |
| 26 | —CH₂CH₂CH₂NH₂ | —H | —CF₃ | —Cl |
| 27 | —(CH₂)₅— | | —CF₃ | —Cl |
| 28 | —(CH₂)₄— | | —CF₃ | —Cl |
| 29 | —(CH₂)₂—O—(CH₂)₂— | | —CF₃ | —Cl |
| 30 | -Ethyl | -Ethyl | —CF₃ | —Cl |
| 31 | -(2-1H-Indol-3-yl)ethyl | —H | —CF₃ | —Cl |
| 32 | -Furan-2-ylmethyl | —H | —CF₃ | —Cl |
| 33 | -Benzyl | —H | —CF₃ | —Cl |
| 34 | -Isobutyl | —H | —CF₃ | —Cl |
| 35 | -(1H-Imidazol-2-yl)methyl | —H | —CF₃ | —H |
| 36 | -Decahydronaphthalen-2-yl | —H | —CF₃ | —H |
| 37 | —H | —H | —CF₃ | -Methyl |

and for each compound of formula (Ia) as set forth above:
the numerals 1 through 7 in formula (Ia) represent carbon atoms;
the carbon atoms at numerals 2, 3 and 5 are independently optionally substituted with —X, —R or —OR;
the carbon atom at numeral 7 is independently substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

9. The method of claim 6 where the nicotine dependence is the result of cigarette smoking.

10. The method of claim 9 where the method results in a reduction of the number of cigarettes smoked by the subject over a period of time.

11. The method of claim 9 where the method results in a cessation of cigarette smoking by the subject.

12. A method of treating nicotine dependence in a subject resulting from the use of tobacco containing products, wherein the method comprises co-administering to the subject a pharmacological smoking reduction therapy and a therapeutically effective amount of a compound of formula (I): 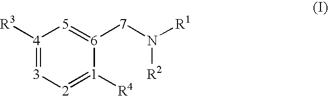
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹ and R² are independently selected from H, optionally substituted aryl, optionally substituted cycloalkyl, and optionally substituted C₁₋₃₀alkyl;
or R¹ and R², together with the nitrogen to which they are attached, form an optionally substituted heterocyclyl ring;
R³ is independently selected from X, optionally substituted C₁₋₃₀alkyl, C₁₋₃₀haloalkyl, nitro, carboxyl, —S(O)₂R, and —S(O)₂N(R)₂;
R⁴ is independently selected from H, X, optionally substituted C₁₋₃₀alkyl, C₁₋₃₀haloalkyl, —OR, —SR and —N(R)₂;
or R⁴ and R², together with the carbon and nitrogen to which they are attached, form an optionally substituted fused heterocyclyl ring;
where the numerals 1 through 7 in the formula (I) represent carbon atoms;
where the carbon atoms at numerals 2, 3 and 5 are independently optionally substituted with —X, —R, —N(R)₂ or —OR;
and where the carbon atom at numeral 7 is independently substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently fluoro, bromo, chloro or iodo; and
each R is independently selected from H and a C₁₋₃₀ organic moiety.

13. The method of claim 12 wherein the compound of formula (I) is a compound of formula (I) wherein:
R¹ and R² are independently selected from H; optionally substituted phenyl; optionally substituted benzyl; optionally substituted cyclopentyl; optionally substituted cyclohexyl; optionally substituted decahydronaphthalenyl; optionally substituted cyclopropylmethyl; optionally substituted cyclohexylmethyl; C₁₋₈alkyl optionally substituted with halo, —OR or —N(R)₂; optionally substituted furanylmethyl; optionally substituted tetrahydrofuranylmethyl; optionally substituted indolylethyl; and optionally substituted imidazolylmethyl;
or R¹ and R², together with the nitrogen to which they are attached, form an optionally substituted pyrrolidinyl, piperidinyl or morpholinyl ring;
R³ is independently selected from X, optionally substituted C₁₋₈alkyl, C₁₋₈haloalkyl, nitro, carboxyl, —S(O)₂R, and —S(O)₂N(R)₂;
R⁴ is independently selected from H, X, optionally substituted C₁₋₈alkyl, C₁₋₈haloalkyl, —OR, —SR and —N(R)₂;
the numerals 1 through 7 in formula (Ia) represent carbon atoms;
the carbon atoms at numerals 2, 3 and 5 are independently optionally substituted with —X, —R or —OR;
the carbon atom at numeral 7 is optionally substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

14. The method of claim 13 wherein the compound of formula (I) is a compound of formula (Ia): 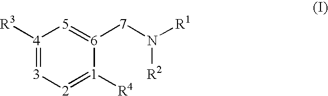
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹, R², R³ and R⁴ are selected as set forth in the following table for each compound of formula (Ia):

| Com- | | | | |
|---|---|---|---|---|
| pound | | | | |
| of | | | | |
| Formula | | | | |
| (Ia) | R¹ | R² | R³ | R⁴ |
| 1 | -Cyclopentyl | —H | —CF₃ | —H |
| 2 | -Isopropyl | -Isopropyl | —CF₃ | —H |
| 3 | -Furan-2-ylmethyl | —H | —CF₃ | —H |
| 4 | -Phenyl | —H | —CF₃ | —H |
| 5 | -Isobutyl | —H | —CF₃ | —H |
| 6 | -Cyclopropylmethyl | —H | —CF₃ | —H |
| 7 | -Benzyl | —H | —CF₃ | —H |
| 8 | -Cyclohexylmethyl | —H | —CF₃ | —H |
| 9 | -Benzyl | -Methyl | —CF₃ | —H |
| 10 | -Tetrahydrofuran-2- | —H | —CF₃ | —H |
| | ylmethyl | | | |
| 11 | —CH₂CH₂OH | —H | —CF₃ | —H |
| 12 | -3-Trifluoromethylbenzyl | —H | —CF₃ | —H |
| 13 | —CH₂CH₂OH | —CH₂CH₂OH | —CF₃ | —H |
| 14 | —CH₂CH₂NH₂ | —H | —CF₃ | —H |
| 15 | -Methyl | —H | —CF₃ | —Cl |
| 16 | -Propyl | —H | —CF₃ | —Cl |
| 17 | —CH₂CH₂OH | —H | —CF₃ | —Cl |
| 18 | -Tetrahydrofuran-2- | —H | —CF₃ | —Cl |
| | ylmethyl | | | |
| 19 | -Cyclohexyl | —H | —CF₃ | —Cl |
| 20 | -Methyl | -Methyl | —CF₃ | —Cl |
| 21 | —CH₂CH₂Cl | —H | —CF₃ | —Cl |
| 22 | —CH₂CH₂NH₂ | —H | —CF₃ | —Cl |
| 23 | -tert-Butyl | —H | —CF₃ | —Cl |
| 24 | -tert-Butyl | —H | —CF₃ | —H |
| 25 | -(2-Chloro-5- | —H | —CF₃ | —Cl |
| | trifluoromethyl)benzyl | | | |
| 26 | —CH₂CH₂CH₂NH₂ | —H | —CF₃ | —Cl |
| 27 | —(CH₂)₅— | | —CF₃ | —Cl |
| 28 | —(CH₂)₄— | | —CF₃ | —Cl |
| 29 | —(CH₂)₂—O—(CH₂)₂— | | —CF₃ | —Cl |
| 30 | -Ethyl | -Ethyl | —CF₃ | —Cl |
| 31 | -(2-1H-Indol-3-yl)ethyl | —H | —CF₃ | —Cl |
| 32 | -Furan-2-ylmethyl | —H | —CF₃ | —Cl |
| 33 | -Benzyl | —H | —CF₃ | —Cl |
| 34 | -Isobutyl | —H | —CF₃ | —Cl |
| 35 | -(1H-Imidazol-2-yl)methyl | —H | —CF₃ | —H |
| 36 | -Decahydronaphthalen-2-yl | —H | —CF₃ | —H |
| 37 | —H | —H | —CF₃ | -Methyl |

and for each compound of formula (Ia) as set forth above:
the numerals 1 through 7 in formula (Ia) represent carbon atoms;
the carbon atoms at numerals 2, 3 and 5 are independently optionally substituted with —X, —R or —OR;
the carbon atom at numeral 7 is independently substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

15. The method of claim 12 where the nicotine dependence is the result of cigarette smoking.

16. The method of claim 15 where the method results in a reduction in the number of cigarettes smoked by the subject over a period of time.

17. The method of claim 15 where the method results in a cessation of cigarette smoking by the subject.

18. A method of treating nicotine dependence in a subject resulting from the use of tobacco containing products, wherein the method comprises administering to the subject behavioral or alternative smoking reduction therapy and administering to the subject a therapeutically effective amount of a compound of formula (I): 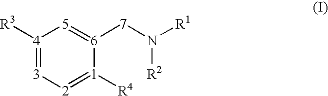
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹ and R² are independently selected from H, optionally substituted aryl, optionally substituted cycloalkyl, and optionally substituted C₁₋₃₀alkyl;
or R¹ and R², together with the nitrogen to which they are attached, form an optionally substituted heterocyclyl ring;
R³ is independently selected from X, optionally substituted C₁₋₃₀alkyl, C₁₋₃₀haloalkyl, nitro, carboxyl, —S(O)₂R, and —S(O)₂N(R)₂;
R⁴ is independently selected from H, X, optionally substituted C₁₋₃₀alkyl, C₁₋₃₀haloalkyl, —OR, —SR and —N(R)₂;
or R⁴ and R², together with the carbon and nitrogen to which they are attached, form an optionally substituted fused heterocyclyl ring;
where the numerals 1 through 7 in the formula (I) represent carbon atoms;
where the carbon atoms at numerals 2, 3 and 5 are independently optionally substituted with —X, —R, —N(R)₂ or —OR;
and where the carbon atom at numeral 7 is independently substituted with:
a) one of the following: ═O, ═C(R)₂, ═C═C(R)₂, —C(R)₂[C(R)₂]ₙ— and —O[C(R)₂]O— wherein n is 1 to 6; or
b) two of the following, which are independently selected: —X, —R and —OR;
each X is independently fluoro, bromo, chloro or iodo; and
each R is independently selected from H and a C₁₋₃₀ organic moiety.

19. The method of claim 18 wherein the compound of formula (I) is a compound of formula (I) wherein:
R¹ and R² are independently selected from H; optionally substituted phenyl; optionally substituted benzyl; optionally substituted cyclopentyl; optionally substituted cyclohexyl; optionally substituted decahydronaphthalenyl; optionally substituted cyclopropylmethyl; optionally substituted cyclohexylmethyl; C₁₋₈alkyl optionally substituted with halo, —OR or —N(R)₂; optionally substituted furanylmethyl; optionally substituted tetrahydrofuranylmethyl; optionally substituted indolylethyl; and optionally substituted imidazolylmethyl;
or R¹ and R², together with the nitrogen to which they are attached, form an optionally substituted pyrrolidinyl, piperidinyl or morpholinyl ring;
R³ is independently selected from X, optionally substituted C₁₋₈alkyl, C₁₋₈haloalkyl, nitro, carboxyl, —S(O)₂R, and —S(O)₂N(R)₂;
R⁴ is independently selected from H, X, optionally substituted C₁₋₈alkyl, C₁₋₈haloalkyl, —OR, —SR and —N(R)₂;
the numerals 1 through 7 in formula (Ia) represent carbon atoms;
the carbon atoms at numerals 2, 3 and 5 are independently optionally substituted with —X, —R or —OR;
the carbon atom at numeral 7 is optionally substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

20. The method of claim 19 wherein the compound of formula (I) is a compound of formula (Ia): 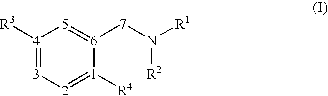
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹, R², R³ and R⁴ are selected as set forth in the following table for each compound of formula (Ia):

| Com- | | | | |
|---|---|---|---|---|
| pound | | | | |
| of | | | | |
| Formula | | | | |
| (Ia) | R¹ | R² | R³ | R⁴ |
| 1 | -Cyclopentyl | —H | —CF₃ | —H |
| 2 | -Isopropyl | -Isopropyl | —CF₃ | —H |
| 3 | -Furan-2-ylmethyl | —H | —CF₃ | —H |
| 4 | -Phenyl | —H | —CF₃ | —H |
| 5 | -Isobutyl | —H | —CF₃ | —H |
| 6 | -Cyclopropylmethyl | —H | —CF₃ | —H |
| 7 | -Benzyl | —H | —CF₃ | —H |
| 8 | -Cyclohexylmethyl | —H | —CF₃ | —H |
| 9 | -Benzyl | -Methyl | —CF₃ | —H |
| 10 | -Tetrahydrofuran-2- | —H | —CF₃ | —H |
| | ylmethyl | | | |
| 11 | —CH₂CH₂OH | —H | —CF₃ | —H |
| 12 | -3-Trifluoromethylbenzyl | —H | —CF₃ | —H |
| 13 | —CH₂CH₂OH | —CH₂CH₂OH | —CF₃ | —H |
| 14 | —CH₂CH₂NH₂ | —H | —CF₃ | —H |
| 15 | -Methyl | —H | —CF₃ | —Cl |
| 16 | -Propyl | —H | —CF₃ | —Cl |
| 17 | —CH₂CH₂OH | —H | —CF₃ | —Cl |
| 18 | -Tetrahydrofuran-2- | —H | —CF₃ | —Cl |
| | ylmethyl | | | |
| 19 | -Cyclohexyl | —H | —CF₃ | —Cl |
| 20 | -Methyl | -Methyl | —CF₃ | —Cl |
| 21 | —CH₂CH₂Cl | —H | —CF₃ | —Cl |
| 22 | —CH₂CH₂NH₂ | —H | —CF₃ | —Cl |
| 23 | -tert-Butyl | —H | —CF₃ | —Cl |
| 24 | -tert-Butyl | —H | —CF₃ | —H |
| 25 | -(2-Chloro-5- | —H | —CF₃ | —Cl |
| | trifluoromethyl)benzyl | | | |
| 26 | —CH₂CH₂CH₂NH₂ | —H | —CF₃ | —Cl |
| 27 | —(CH₂)₅— | | —CF₃ | —Cl |
| 28 | —(CH₂)₄— | | —CF₃ | —Cl |
| 29 | —(CH₂)₂—O—(CH₂)₂— | | —CF₃ | —Cl |
| 30 | -Ethyl | -Ethyl | —CF₃ | —Cl |
| 31 | -(2-1H-Indol-3-yl)ethyl | —H | —CF₃ | —Cl |
| 32 | -Furan-2-ylmethyl | —H | —CF₃ | —Cl |
| 33 | -Benzyl | —H | —CF₃ | —Cl |
| 34 | -Isobutyl | —H | —CF₃ | —Cl |
| 35 | -(1H-Imidazol-2-yl)methyl | —H | —CF₃ | —H |
| 36 | -Decahydronaphthalen-2-yl | —H | —CF₃ | —H |
| 37 | —H | —H | —CF₃ | -Methyl |

and for each compound of formula (Ia) as set forth above:
the numerals 1 through 7 in formula (Ia) represent carbon atoms;
the carbon atoms at numerals 2, 3 and 5 are independently optionally substituted with —X, —R or —OR;
the carbon atom at numeral 7 is independently substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

21. The method of claim 18 where the nicotine dependence is the result of cigarette smoking.

22. The method of claim 21 where the method results in a reduction in the number of cigarettes smoked by the subject over a period of time.

23. The method of claim 21 where the objective or result is cessation of cigarette smoking.

24. A method of reducing exposure of a subject to compounds resulting from the use of tobacco products, wherein the method comprises administering to the subject a therapeutically effective amount of a compound of formula (I): 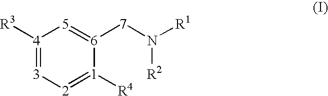
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹ and R² are independently selected from H, optionally substituted aryl, optionally substituted cycloalkyl, and optionally substituted C₁₋₃₀alkyl;
or R¹ and R², together with the nitrogen to which they are attached, form an optionally substituted heterocyclyl ring;
R³ is independently selected from X, optionally substituted C₁₋₃₀alkyl, C₁₋₃₀haloalkyl, nitro, carboxyl, —S(O)₂R, and —S(O)₂N(R)₂;
R⁴ is independently selected from H, X, optionally substituted C₁₋₃₀alkyl, C₁₋₃₀haloalkyl, —OR, —SR and —N(R)₂;
or R⁴ and R², together with the carbon and nitrogen to which they are attached, form an optionally substituted fused heterocyclyl ring;
where the numerals 1 through 7 in the formula (I) represent carbon atoms;
where the carbon atoms at numerals 2, 3 and 5 are independently optionally substituted with —X, —R, —N(R)₂ or —OR;
and where the carbon atom at numeral 7 is independently substituted with:
a) one of the following: ═O, ═C(R)₂, ═C═C(R)₂, —C(R)₂[C(R)₂]ₙ— and —O[C(R)₂]O— wherein n is 1 to 6; or
b) two of the following, which are independently selected: —X, —R and —OR;
each X is independently fluoro, bromo, chloro or iodo; and
each R is independently selected from H and a C₁₋₃₀ organic moiety.

25. The method of claim 24 wherein the compound of formula (I) is a compound of formula (I) wherein:
R¹ and R² are independently selected from H; optionally substituted phenyl; optionally substituted benzyl; optionally substituted cyclopentyl; optionally substituted cyclohexyl; optionally substituted decahydronaphthalenyl; optionally substituted cyclopropylmethyl; optionally substituted cyclohexylmethyl; C₁₋₈alkyl optionally substituted with halo, —OR or —N(R)₂; optionally substituted furanylmethyl; optionally substituted tetrahydrofuranylmethyl; optionally substituted indolylethyl; and optionally substituted imidazolylmethyl;
or R¹ and R², together with the nitrogen to which they are attached, form an optionally substituted pyrrolidinyl, piperidinyl or morpholinyl ring;
R³ is independently selected from X, optionally substituted C₁₋₈alkyl, C₁₋₈haloalkyl, nitro, carboxyl, —S(O)₂R, and —S(O)₂N(R)₂;
R⁴ is independently selected from H, X, optionally substituted C₁₋₈alkyl, C₁₋₈haloalkyl, —OR, —SR and —N(R)₂;
the numerals 1 through 7 in formula (Ia) represent carbon atoms;
the carbon atoms at numerals 2, 3 and 5 are independently optionally substituted with —X, —R or —OR;
the carbon atom at numeral 7 is optionally substituted with:
a) one of the following: ═O, ═C(R)₂, ═C═C(R)₂, —C(R)₂[C(R)₂]ₙ— and —O[C(R)₂]O— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

26. The method of claim 25 wherein the compound of formula (I) is a compound of formula (Ia): 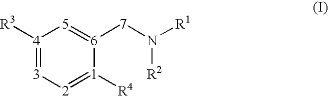
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹, R², R³ and R⁴ are selected as set forth in the following table for each compound of formula (Ia):

| Com- | | | | |
|---|---|---|---|---|
| pound | | | | |
| of | | | | |
| Formula | | | | |
| (Ia) | R¹ | R² | R³ | R⁴ |
| 1 | -Cyclopentyl | —H | —CF₃ | —H |
| 2 | -Isopropyl | -Isopropyl | —CF₃ | —H |
| 3 | -Furan-2-ylmethyl | —H | —CF₃ | —H |
| 4 | -Phenyl | —H | —CF₃ | —H |
| 5 | -Isobutyl | —H | —CF₃ | —H |
| 6 | -Cyclopropylmethyl | —H | —CF₃ | —H |
| 7 | -Benzyl | —H | —CF₃ | —H |
| 8 | -Cyclohexylmethyl | —H | —CF₃ | —H |
| 9 | -Benzyl | -Methyl | —CF₃ | —H |
| 10 | -Tetrahydrofuran-2- | —H | —CF₃ | —H |
| | ylmethyl | | | |
| 11 | —CH₂CH₂OH | —H | —CF₃ | —H |
| 12 | -3-Trifluoromethylbenzyl | —H | —CF₃ | —H |
| 13 | —CH₂CH₂OH | —CH₂CH₂OH | —CF₃ | —H |
| 14 | —CH₂CH₂NH₂ | —H | —CF₃ | —H |
| 15 | -Methyl | —H | —CF₃ | —Cl |
| 16 | -Propyl | —H | —CF₃ | —Cl |
| 17 | —CH₂CH₂OH | —H | —CF₃ | —Cl |
| 18 | -Tetrahydrofuran-2- | —H | —CF₃ | —Cl |
| | ylmethyl | | | |
| 19 | -Cyclohexyl | —H | —CF₃ | —Cl |
| 20 | -Methyl | -Methyl | —CF₃ | —Cl |
| 21 | —CH₂CH₂Cl | —H | —CF₃ | —Cl |
| 22 | —CH₂CH₂NH₂ | —H | —CF₃ | —Cl |
| 23 | -tert-Butyl | —H | —CF₃ | —Cl |
| 24 | -tert-Butyl | —H | —CF₃ | —H |
| 25 | -(2-Chloro-5- | —H | —CF₃ | —Cl |
| | trifluoromethyl)benzyl | | | |
| 26 | —CH₂CH₂CH₂NH₂ | —H | —CF₃ | —Cl |
| 27 | —(CH₂)₅— | | —CF₃ | —Cl |
| 28 | —(CH₂)₄— | | —CF₃ | —Cl |
| 29 | —(CH₂)₂—O—(CH₂)₂— | | —CF₃ | —Cl |
| 30 | -Ethyl | -Ethyl | —CF₃ | —Cl |
| 31 | -(2-1H-Indol-3-yl)ethyl | —H | —CF₃ | —Cl |
| 32 | -Furan-2-ylmethyl | —H | —CF₃ | —Cl |
| 33 | -Benzyl | —H | —CF₃ | —Cl |
| 34 | -Isobutyl | —H | —CF₃ | —Cl |
| 35 | -(1H-Imidazol-2-yl)methyl | —H | —CF₃ | —H |
| 36 | -Decahydronaphthalen-2-yl | —H | —CF₃ | —H |
| 37 | —H | —H | —CF₃ | -Methyl |

and for each compound of formula (Ia) as set forth above:
the numerals 1 through 7 in formula (Ia) represent carbon atoms;
the carbon atoms at numerals 2, 3 and 5 are independently optionally substituted with —X, —R or —OR;
the carbon atom at numeral 7 is independently substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

27. The method of claim 24 where the compounds resulting from the use of tobacco products are carcinogens or other physiologically harmful compounds.

28. The method of claim 27 where the method results in a reduction in the amount of tobacco product used by the subject.

29. The method of claim 27 where the method results in a cessation of use of tobacco products by the subject.

30. The method of claim 24 where the compounds are procarcinogens or precursors to carcinogens or other physiologically harmful compounds.

31. The method of claim 30 where the method results in the inhibition of the conversion of the procarginogen or the precursor to a carcinogen or other physiologically harmful compound.

32. A pharmaceutical composition comprising a pharmaceutically acceptable carrier and a compound of formula (I): 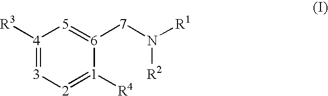
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹ and R² are independently selected from H, optionally substituted aryl, optionally substituted cycloalkyl, and optionally substituted C₁₋₃₀alkyl;
or R¹ and R², together with the nitrogen to which they are attached, form an optionally substituted heterocyclyl ring;
R³ is independently selected from X, optionally substituted C₁₋₃₀alkyl, C₁₋₃₀haloalkyl, nitro, carboxyl, —S(O)₂R, and —S(O)₂N(R)₂;
R⁴ is independently selected from H, X, optionally substituted C₁₋₃₀alkyl, C₁₋₃₀haloalkyl, —OR, —SR and —N(R)₂;
or R⁴ and R², together with the carbon and nitrogen to which they are attached, form an optionally substituted fused heterocyclyl ring;
where the numerals 1 through 7 in the formula (I) represent carbon atoms;
where the carbon atoms at numerals 2, 3 and 5 are independently optionally substituted with —X, —R, —N(R)₂ or —OR;
and where the carbon atom at numeral 7 is independently substituted with:
a) one of the following: ═O, ═C(R)₂, ═C═C(R)₂, —C(R)₂[C(R)₂]ₙ— and —O[C(R)₂]O— wherein n is 1 to 6; or
b) two of the following, which are independently selected: —X, —R and —OR;
each X is independently fluoro, bromo, chloro or iodo; and
each R is independently selected from H and a C₁₋₃₀ organic moiety.

33. The method of claim 32 wherein the compound of formula (I) is a compound of formula (I) wherein:
R¹ and R² are independently selected from H; optionally substituted phenyl; optionally substituted benzyl; optionally substituted cyclopentyl; optionally substituted cyclohexyl; optionally substituted decahydronaphthalenyl; optionally substituted cyclopropylmethyl; optionally substituted cyclohexylmethyl; C₁₋₈alkyl optionally substituted with halo, —OR or —N(R)₂; optionally substituted furanylmethyl; optionally substituted tetrahydrofuranylmethyl; optionally substituted indolylethyl; and optionally substituted imidazolylmethyl;
or R¹ and R², together with the nitrogen to which they are attached, form an optionally substituted pyrrolidinyl, piperidinyl or morpholinyl ring;
R³ is independently selected from X, optionally substituted C₁₋₈alkyl, C₁₋₈haloalkyl, nitro, carboxyl, —S(O)₂R, and —S(O)₂N(R)₂;
R⁴ is independently selected from H, X, optionally substituted C₁₋₈alkyl, C₁₋₈haloalkyl, —OR, —SR and —N(R)₂;
the numerals 1 through 7 in formula (Ia) represent carbon atoms;
the carbon atoms at numerals 2, 3 and 5 are independently optionally substituted with —X, —R or —OR;
the carbon atom at numeral 7 is optionally substituted with:
a) one of the following: ═O, ═C(R)₂, ═C═C(R)₂, —C(R)₂[C(R)₂]ₙ— and —O[C(R)₂]O— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

34. The pharmaceutical composition of claim 33 wherein the compound of formula (I) is a compound of formula (Ia): 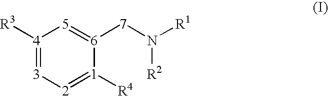
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹, R², R³ and R⁴ are selected as set forth in the following table for each compound of formula (Ia):

| Com- | | | | |
|---|---|---|---|---|
| pound | | | | |
| of | | | | |
| Formula | | | | |
| (Ia) | R¹ | R² | R³ | R⁴ |
| 1 | -Cyclopentyl | —H | —CF₃ | —H |
| 2 | -Isopropyl | -Isopropyl | —CF₃ | —H |
| 3 | -Furan-2-ylmethyl | —H | —CF₃ | —H |
| 4 | -Phenyl | —H | —CF₃ | —H |
| 5 | -Isobutyl | —H | —CF₃ | —H |
| 6 | -Cyclopropylmethyl | —H | —CF₃ | —H |
| 7 | -Benzyl | —H | —CF₃ | —H |
| 8 | -Cyclohexylmethyl | —H | —CF₃ | —H |
| 9 | -Benzyl | -Methyl | —CF₃ | —H |
| 10 | -Tetrahydrofuran-2- | —H | —CF₃ | —H |
| | ylmethyl | | | |
| 11 | —CH₂CH₂OH | —H | —CF₃ | —H |
| 12 | -3-Trifluoromethylbenzyl | —H | —CF₃ | —H |
| 13 | —CH₂CH₂OH | —CH₂CH₂OH | —CF₃ | —H |
| 14 | —CH₂CH₂NH₂ | —H | —CF₃ | —H |
| 15 | -Methyl | —H | —CF₃ | —Cl |
| 16 | -Propyl | —H | —CF₃ | —Cl |
| 17 | —CH₂CH₂OH | —H | —CF₃ | —Cl |
| 18 | -Tetrahydrofuran-2- | —H | —CF₃ | —Cl |
| | ylmethyl | | | |
| 19 | -Cyclohexyl | —H | —CF₃ | —Cl |
| 20 | -Methyl | -Methyl | —CF₃ | —Cl |
| 21 | —CH₂CH₂Cl | —H | —CF₃ | —Cl |
| 22 | —CH₂CH₂NH₂ | —H | —CF₃ | —Cl |
| 23 | -tert-Butyl | —H | —CF₃ | —Cl |
| 24 | -tert-Butyl | —H | —CF₃ | —H |
| 25 | -(2-Chloro-5- | —H | —CF₃ | —Cl |
| | trifluoromethyl)benzyl | | | |
| 26 | —CH₂CH₂CH₂NH₂ | —H | —CF₃ | —Cl |
| 27 | —(CH₂)₅— | | —CF₃ | —Cl |
| 28 | —(CH₂)₄— | | —CF₃ | —Cl |
| 29 | —(CH₂)₂—O—(CH₂)₂— | | —CF₃ | —Cl |
| 30 | -Ethyl | -Ethyl | —CF₃ | —Cl |
| 31 | -(2-1H-Indol-3-yl)ethyl | —H | —CF₃ | —Cl |
| 32 | -Furan-2-ylmethyl | —H | —CF₃ | —Cl |
| 33 | -Benzyl | —H | —CF₃ | —Cl |
| 34 | -Isobutyl | —H | —CF₃ | —Cl |
| 35 | -(1H-Imidazol-2-yl)methyl | —H | —CF₃ | —H |
| 36 | -Decahydronaphthalen-2-yl | —H | —CF₃ | —H |
| 37 | —H | —H | —CF₃ | -Methyl |

and for each compound of formula (Ia) as set forth above:
the numerals 1 through 7 in formula (Ia) represent carbon atoms;
the carbon atoms at numerals 2, 3 and 5 are independently optionally substituted with —X, —R or —OR;
the carbon atom at numeral 7 is independently substituted with:
a) one of the following: ═O, ═C(R)₂, ═C═C(R)₂, —C(R)₂[C(R)₂]ₙ— and —O[C(R)₂]ₙO— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

35. A compound of formula (Ia): 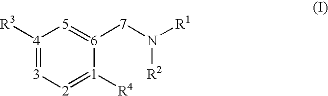
as isolated stereoisomers or mixtures thereof, or pharmaceutically acceptable salts, solvates, stereoisomers and prodrugs thereof, in isolation or in mixture, with or without a pharmaceutically acceptable carrier, diluent or excipient;
wherein:
R¹, R², R³ and R⁴ are selected as set forth in the following table for each compound of formula (Ia):

| Com- | | | | |
|---|---|---|---|---|
| pound | | | | |
| of | | | | |
| Formula | | | | |
| (Ia) | R¹ | R² | R³ | R⁴ |
| 1 | -Cyclopentyl | —H | —CF₃ | —H |
| 2 | -Isopropyl | -Isopropyl | —CF₃ | —H |
| 3 | -Furan-2-ylmethyl | —H | —CF₃ | —H |
| 4 | -Phenyl | —H | —CF₃ | —H |
| 5 | -Isobutyl | —H | —CF₃ | —H |
| 6 | -Cyclopropylmethyl | —H | —CF₃ | —H |
| 7 | -Benzyl | —H | —CF₃ | —H |
| 8 | -Cyclohexylmethyl | —H | —CF₃ | —H |
| 9 | -Benzyl | -Methyl | —CF₃ | —H |
| 10 | -Tetrahydrofuran-2- | —H | —CF₃ | —H |
| | ylmethyl | | | |
| 11 | —CH₂CH₂OH | —H | —CF₃ | —H |
| 12 | -3-Trifluoromethylbenzyl | —H | —CF₃ | —H |
| 13 | —CH₂CH₂OH | —CH₂CH₂OH | —CF₃ | —H |
| 14 | —CH₂CH₂NH₂ | —H | —CF₃ | —H |
| 15 | -Methyl | —H | —CF₃ | —Cl |
| 16 | -Propyl | —H | —CF₃ | —Cl |
| 17 | —CH₂CH₂OH | —H | —CF₃ | —Cl |
| 18 | -Tetrahydrofuran-2- | —H | —CF₃ | —Cl |
| | ylmethyl | | | |
| 19 | -Cyclohexyl | —H | —CF₃ | —Cl |
| 20 | -Methyl | -Methyl | —CF₃ | —Cl |
| 21 | —CH₂CH₂Cl | —H | —CF₃ | —Cl |
| 22 | —CH₂CH₂NH₂ | —H | —CF₃ | —Cl |
| 23 | -tert-Butyl | —H | —CF₃ | —Cl |
| 24 | -tert-Butyl | —H | —CF₃ | —H |
| 25 | -(2-Chloro-5- | —H | —CF₃ | —Cl |
| | trifluoromethyl)benzyl | | | |
| 26 | —CH₂CH₂CH₂NH₂ | —H | —CF₃ | —Cl |
| 27 | —(CH₂)₅— | | —CF₃ | —Cl |
| 28 | —(CH₂)₄— | | —CF₃ | —Cl |
| 29 | —(CH₂)₂—O—(CH₂)₂— | | —CF₃ | —Cl |
| 30 | -Ethyl | -Ethyl | —CF₃ | —Cl |
| 31 | -(2-1H-Indol-3-yl)ethyl | —H | —CF₃ | —Cl |
| 32 | -Furan-2-ylmethyl | —H | —CF₃ | —Cl |
| 33 | -Benzyl | —H | —CF₃ | —Cl |
| 34 | -Isobutyl | —H | —CF₃ | —Cl |
| 35 | -(1H-Imidazol-2-yl)methyl | —H | —CF₃ | —H |
| 36 | -Decahydronaphthalen-2-yl | —H | —CF₃ | —H |
| 37 | —H | —H | —CF₃ | -Methyl |

and for each compound of formula (Ia) as set forth above:
the numerals 1 through 7 in formula (Ia) represent carbon atoms;
the carbon atoms at numerals 2, 3 and 5 are independently optionally substituted with —X, —R or —OR;
the carbon atom at numeral 7 is independently substituted with:
a) one of the following: ═O, ═C(R)₂, ═C═C(R)₂, —C(R)₂[C(R)₂]ₙ— and —O[C(R)₂]O— wherein n is 1 to 6; or
b) two of the following, which are independently selected: —X, —R and —OR;
each X is independently selected from fluoro, bromo, chloro or iodo; and
each R is independently selected from H, alkyl, alkenyl, haloalkyl, cycloalkyl, cycloalkylalkyl, aryl, aralkyl, aralkenyl, heterocyclyl or heterocyclylalkyl.

36. The compound of claim 35 wherein:
the carbon atoms at numerals 2, 3 and 5 are substituted with H; and
the carbon atom at numeral 7 is substituted with two H's.
